(19) United States
(12) Patent Application Publication
(10) Pub. No.: US 2010/0144710 A1
(43) Pub. Date: Jun. 10, 2010

(54) Piperazine and Piperidine MGLUR5 Potentiators

(75) Inventors: Jalaj Arora, Ontario (CA); Methvin Isaac, Ontario (CA); Abdelmalik Slassi, Ontario (CA); Louise Edwards, Ontario (CA); Satheesh Nair, San Ramon (CA); Fupeng Ma, Melrose, MA (US)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: ASTRAZENECA AB, Sodertalje (SE)

(21) Appl. No.: 12/530,351

(22) PCT Filed: Feb. 28, 2008
(86) PCT No.: PCT/US2008/055261

§ 371 Date: Feb. 24, 2010

Related U.S. Application Data
(60) Provisional application No. 60/894,114, filed on Mar. 9, 2007.

(51) Int. Cl. A61K 31/496; C07D 401/04; C07D 403/04; A61K 31/551; A61P 25/18; A61P 25/00

(52) U.S. Cl. 514/218; 544/360; 544/357; 540/575; 514/253.01; 514/252.11

(57) ABSTRACT

Compounds of Formula I or pharmaceutically acceptable salts or solvates thereof, wherein A, B, D, Ar1, Ar2, R2, R3, R4, a, m and n are defined in the specification, methods for the use thereof, processes for making and pharmaceutical compositions containing the same.

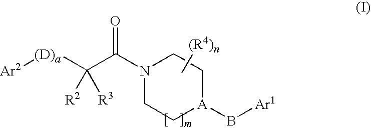

### FIELD OF THE INVENTION

The present invention relates to a new class of compounds, to pharmaceutical formulations containing said compounds and to the use of said compounds in therapy. The invention further relates to the process for the preparation of said compounds and to new intermediates prepared therein.

### BACKGROUND OF THE INVENTION

Glutamate is the major excitatory neurotransmitter in the mammalian central nervous system (CNS). Glutamate produces its effects on central neurons by binding to and thereby activating cell surface receptors. These receptors have been divided into two major classes, the ionotropic and metabotropic glutamate receptors, based on the structural features of the receptor proteins, the means by which the receptors transduce signals into the cell, and pharmacological profiles.

The metabotropic glutamate receptors (mGluRs) are G protein-coupled receptors that activate a variety of intracellular second messenger systems following the binding of glutamate. Activation of mGluRs in intact mammalian neurons elicits one or more of the following responses: activation of phospholipase C; increases in phosphoinositide (PI) hydrolysis; intracellular calcium release; activation of phospholipase D; activation or inhibition of adenyl cyclase; increases or decreases in the formation of cyclic adenosine monophosphate (cAMP); activation of guanylyl cyclase; increases in the formation of cyclic guanosine monophosphate (cGMP); activation of phospholipase A₂; increases in arachidonic acid release; and increases or decreases in the activity of voltage- and ligand-gated ion channels. Schoepp et al., Trends Pharmacol. Sci. 14:13 (1993), Schoepp, Neurochem. Int. 24:439 (1994), Pin et al., Neuropharmacology 34:1 (1995), Bordi and Ugolini, Prog. Neurobiol. 59:55 (1999).

Molecular cloning has identified eight distinct mGluR subtypes, termed mGluR1 through mGluR8. Nakanishi, Neuron 13:1031 (1994), Pin et al., Neuropharmacology 34:1 (1995), Knopfel et al., J. Med. Chem. 38:1417 (1995). Further receptor diversity occurs via expression of alternatively spliced forms of certain mGluR subtypes. Pin et al., PNAS 89:10331 (1992), Minakami et al., BBRC 199:1136 (1994), Joly et al., J. Neurosci. 15:3970 (1995).

Metabotropic glutamate receptor subtypes may be subdivided into three groups, Group I, Group II, and Group III mGluRs, based on amino acid sequence homology, the second messenger systems utilized by the receptors, and by their pharmacological characteristics. Group I mGluR comprises mGluR1, mGluR5 and their alternatively spliced variants. The binding of agonists to these receptors results in the activation of phospholipase C and the subsequent mobilization of intracellular calcium.

Recent advances in the elucidation of the neurophysiological roles of mGluRs have established these receptors as promising drug targets in the therapy of acute and chronic neurological and psychiatric disorders and chronic and acute pain disorders. Because of the physiological and pathophysiological significance of the mGluRs, there is a need for new drugs and compounds that can modulate mGluR function.

Neurological, psychiatric and pain disorders.

Attempts at elucidating the physiological roles of Group I mGluRs suggest that activation of these receptors elicits neuronal excitation. Various studies have demonstrated that Group I mGluRs agonists can produce postsynaptic excitation upon application to neurons in the hippocampus, cerebral cortex, cerebellum, and thalamus, as well as other CNS regions. Evidence indicates that this excitation is due to direct activation of postsynaptic mGluRs, but it also has been suggested that activation of presynaptic mGluRs occurs, resulting in increased neurotransmitter release. Baskys, Trends Pharmacol. Sci. 15:92 (1992), Schoepp, Neurochem. Int. 24:439 (1994), Pin et al., Neuropharmacology 34:1 (1995), Watkins et al., Trends Pharmacol. Sci. 15:33 (1994).

Metabotropic glutamate receptors have been implicated in a number of normal processes in the mammalian CNS. Activation of mGluRs has been shown to be required for induction of hippocampal long-term potentiation and cerebellar long-term depression, Bashir et al., Nature 363:347 (1993), Bortolotto et al., Nature 368:740 (1994), Aiba et al., Cell 79:365 (1994), Aiba et al., Cell 79:377 (1994). A role for mGluR activation in nociception and analgesia also has been demonstrated, Meller et al., Neuroreport 4: 879 (1993), Bordi and Ugolini, Brain Res. 871:223 (1999). In addition, mGluR activation has been suggested to play a modulatory role in a variety of other normal processes including synaptic transmission, neuronal development, apoptotic neuronal death, synaptic plasticity, spatial learning, olfactory memory, central control of cardiac activity, waking, motor control and control of the vestibulo-ocular reflex. Nakanishi, Neuron 13: 1031 (1994), Pin et al., Neuropharmacology 34:1, Knopfel et al., J. Med. Chem. 38:1417 (1995).

Further, Group I metabotropic glutamate receptors and mGluR5 in particular, have been suggested to play roles in a variety of pathophysiological processes and disorders affecting the CNS. These include stroke, head trauma, anoxic and ischemic injuries, hypoglycemia, epilepsy, neurodegenerative disorders such as Alzheimer's disease and pain. Schoepp et al., Trends Pharmacol. Sci. 14:13 (1993), Cunningham et al., Life Sci. 54:135 (1994), Hollman et al., Ann. Rev. Neurosci. 17:31 (1994), Pin et al., Neuropharmacology 34:1 (1995), Knopfel et al., J. Med. Chem. 38:1417 (1995), Spooren et al., Trends Pharmacol. Sci. 22:331 (2001), Gasparini et al. Curr. Opin. Pharmacol. 2:43 (2002), Neugebauer Pain 98:1 (2002). Much of the pathology in these conditions is thought to be due to excessive glutamate-induced excitation of CNS neurons. Because Group I mGluRs appear to increase glutamate-mediated neuronal excitation via postsynaptic mechanisms and enhanced presynaptic glutamate release, their activation probably contributes to the pathology. Accordingly, selective antagonists of Group I mGluR receptors could be therapeutically beneficial, specifically as neuroprotective agents, analgesics or anticonvulsants.

Further, it has also been shown that mGluR5 antagonists are useful for the treatment of addictions or cravings (for drugs, tobacco, alcohol, any appetizing macronutrients or non-essential food items).

Recent advances in the elucidation of the neurophysiological roles of metabotropic glutamate receptors generally and Group I in particular, have established these receptors as promising drug targets in the therapy of acute and chronic neurological and psychiatric disorders and chronic and acute pain disorders.

### Medical Use

The group I receptor, mGluR5, has been implicated in a number of central nervous system disease states, including pain (Salt and Binns, 2000; Bhave, et al., 2001), anxiety (Spooren, et al., 2000; Tatarczynska, et al., 2001), addiction to cocaine (Chiamulera, et al., 2001) and schizophrenia (Chavez-Noriega, et al., 2002). The N-methyl-D-aspartate (NMDA) receptor, an ionotropic glutamate receptor, has also been implicated in physiological and pathological processes. Of specific interest, blockade of NMDA receptors produces a transient state of psychosis and schizophrenia-like cognitive deficits (Krystal, et al., Arch Gen Psychiatry, 51: 199-214, 1994; Lahti, et al., Neuropsychopharmacol., 13: 9-19, 1995; Newcomer, et al., Neuropsychopharmacol., 20:106-118, 1999). Pharmacological manipulation of NMDA receptor function may be critical for the treatment of many neurological and psychiatric disorders such as epilepsy, Alzheimer's disease, drug dependence and schizophrenia (Kemp and McKernan, 2002). A functional interaction between NMDA receptors and mGluR5 has been demonstrated at a cellular level and at a behavioral level. Thus, activation of Group I mGluRs by DHPG enhanced NMDA-receptor mediated responses in mouse CA1 pyramidal neurones (Mannaioni, et al., J. Neurosci., 21:5925-5934, 2001). This effect was inhibited by MPEP, demonstrating that NMDA receptor function was enhanced through activation of mGluR5 (Mannaioni, et al., J. Neurosci., 21:5925-5934, 2001). Modulation of mGluR5 also altered the cognitive and behavioral abnormalities associated with NMDA receptor deficiency (Homayoun, et al., Neuropsychopharmacol., 29: 1259-1269, 2004). Together these data suggest that potentiation of mGluR5 could be beneficial in the treatment of disorders such as schizophrenia.

### Non-Medical Use

In addition to their use in therapeutic medicine, the compounds of Formula I, as well as salts and hydrates of such compounds, are useful as pharmacological tools in the development and standardization of in vitro and in vivo test systems for the evaluation of the effects of potentiators of mGluR related activity in laboratory animals such as cats, dogs, rabbits, monkeys, rats and mice, as part of the search for new therapeutic agents.

### SUMMARY OF THE INVENTION

It has been discovered that the compounds of the present invention are potentiators of mGluR5 receptor function and are, therefore, useful in the treatment of neurological and psychiatric disorders associated with glutamate dysfunction.

One embodiment of the invention relates to compounds of Formula I or a pharmaceutically acceptable salt or solvate thereof:

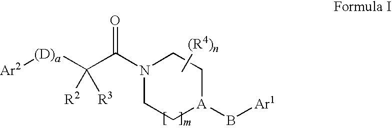

wherein:
Ar¹ is selected from the group consisting of aryl and heteroaryl, substituted with a CN group at the position alpha to the link with group B, further optionally-substituted with one or more substituents selected from the group consisting of alkyl, haloalkyl and halo;
Ar² is selected from the group consisting of aryl and heteroaryl, optionally substituted with one or more substituents selected from the group consisting of halo, alkyl, haloalkyl, alkoxy, haloalkoxy, NR⁵R⁶, O-alkylene-aryl, O-alkylene-heteroaryl, O-alkylene-O-alkyl, cycloalkyl, O-heterocycloalkyl, wherein any cyclic group may be further substituted with one or more substituents selected from the group consisting of alkyl and halo;
A is selected from the group consisting of C and N;
B is a bond when A is N and is group NR when A is C;
D is selected from the group consisting of NR¹ and O;
a is selected from the group consisting of 0 and 1;
m is selected from the group consisting of 1 and 2;
n is selected from the group consisting of 1, 2, 3 and 4;
R, R², R³, R⁵ and R⁶ are independently selected from the group consisting of H and alkyl;
R¹ is selected from the group consisting of H, alkyl, COR, CO₂R and SO₂R; and
R⁴ is selected from the group consisting of H, halo, CN, alkyl, haloalkyl, CH₂OR and CO₂R;
or a pharmaceutically acceptable salt, hydrate, solvate, optical isomer, or combination thereof.

Another embodiment of the invention is a pharmaceutical composition comprising as active ingredient a therapeutically effective amount of the compound according to Formula I and one or more pharmaceutically acceptable diluents, excipients and/or inert carriers.

Other embodiments of the invention, as described in more detail below, relate to a compound according to Formula I for use in therapy, in the treatment of mGluR5 mediated disorders, and in the manufacture of a medicament for the treatment of mGluR5 mediated disorders.

Still other embodiments relate to a method of treatment of mGluR5-mediated disorders, comprising administering to a mammal a therapeutically effective amount of the compound according to Formula I.

### DETAILED DESCRIPTION OF THE PREFERRED EMBODIMENTS

The present invention is based upon the discovery of compounds which are potentiators of metabotropic glutamate receptor function. More particularly, the compounds of the present invention exhibit activity as potentiators of mGluR5 receptor function and, therefore, are useful in therapy, in particular for the treatment of neurological and psychiatric disorders,

### DEFINITIONS

Unless specified otherwise within this specification, the nomenclature used in this specification generally follows the examples and rules stated in Nomenclature of Organic Chemistry, Sections A, B, C, D, E, F, and H, Pergamon Press, Oxford, 1979, which is incorporated by references herein for its exemplary chemical structure names and rules on naming chemical structures. Optionally, a name of a compound may be generated using a chemical naming program: ACD/ChemSketch, Version 5.09/September 2001, Advanced Chemistry Development, Inc., Toronto, Canada,

The term “alkyl” as used herein means a straight- or branched-chain hydrocarbon radical having from one to six carbon atoms, and includes methyl, ethyl, propyl, isopropyl, t-butyl and the like.

The term “alkoxy” as used herein means a straight- or branched-chain alkoxy radical having from one to six carbon atoms and includes methoxy, ethoxy, propyloxy, isopropyloxy, t-butoxy and the like.

The term “halo” as used herein means halogen and includes fluoro, chloro, bromo, iodo and the like, in both radioactive and non-radioactive forms.

The term “haloalkyl” as used herein means an alkyl group in which at least one H atom has been replaced by a halo atom, and includes groups such as CF₃, CH₂Br and the like.

The term “alkylene” as used herein means a difunctional branched or unbranched saturated hydrocarbon radical having one to six carbon atoms, and includes methylene, ethylene, n-propylene, n-butylene and the like.

The term “aryl” as used herein means an aromatic group having five to twelve atoms, and includes phenyl, naphthyl and the like.

The term “heteroaryl” means an aromatic group having from 5 to 8 atoms which includes at least one heteroatom selected from the group consisting of N, S and O, and includes pyridyl, furyl, thienyl, thiazolyl, pyrazinyl, pyrimidinyl, oxazolyl and the like.

The term “pharmaceutically acceptable salt” means either an acid addition salt or a basic addition salt that is compatible with the treatment of patients.

A “pharmaceutically acceptable acid addition salt” is any non-toxic organic or inorganic acid addition salt of the base compounds represented by Formula I, or any of its intermediates. Illustrative inorganic acids which form suitable salts include hydrochloric, hydrobromic, sulfuric and phosphoric acid and acid metal salts such as sodium monohydrogen orthophosphate and potassium hydrogen sulfate. Illustrative organic acids that form suitable salts include the mono-, di- and tricarboxylic acids. Illustrative of such acids are, for example, acetic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, salicylic, 2-phenoxybenzoic, p-toluenesulfonic acid and other sulfonic acids such as methanesulfonic acid and 2-hydroxyethanesulfonic acid. Either the mono- or di-acid salts can be formed, and such salts can exist in either a hydrated, solvated or substantially anhydrous form. In general, the acid addition salts of these compounds are more soluble in water and various hydrophilic organic solvents, and generally demonstrate higher melting points in comparison to their free base forms. The selection criteria for the appropriate salt will be known to one skilled in the art. Other non-pharmaceutically acceptable salts e.g. oxalates may be used for example in the isolation of compounds of Formula I for laboratory use, or for subsequent conversion to a pharmaceutically acceptable acid addition salt.

A “pharmaceutically acceptable basic addition salt” is any non-toxic organic or inorganic base addition salt of the acid compounds represented by Formula I or any of its intermediates. Illustrative inorganic bases which form suitable salts include lithium, sodium, potassium, calcium, magnesium or barium hydroxides. Illustrative organic bases which form suitable salts include aliphatic, alicyclic or aromatic organic amines such as methylamine, trimethyl amine and picoline or ammonia. The selection of the appropriate salt may be important so that an ester functionality, if any, elsewhere in the molecule is not hydrolyzed. The selection criteria for the appropriate salt will be known to one skilled in the art.

“Solvate” means a compound of Formula I or the pharmaceutically acceptable salt of a compound of Formula I wherein molecules of a suitable solvent are incorporated in a crystal lattice. A suitable solvent is physiologically tolerable at the dosage administered as the solvate. Examples of suitable solvents are ethanol, water and the like. When water is the solvent, the molecule is referred to as a hydrate.

The term “stereoisomers” is a general term for all isomers of the individual molecules that differ only in the orientation of their atoms in space. It includes minor image isomers (enantiomers), geometric (cis/trans) isomers and isomers of compounds with more than one chiral centre that are not mirror images of one another (diastereomers).

The term “treat” or “treating” means to alleviate symptoms, eliminate the causation of the symptoms either on a temporary or permanent basis, or to prevent or slow the appearance of symptoms of the named disorder or condition.

The term “therapeutically effective amount” means an amount of the compound of Formula I that is effective in treating the named disorder or condition.

The term “pharmaceutically acceptable carrier” means a non-toxic solvent, dispersant, excipient, adjuvant or other material which is mixed with the active ingredient in order to permit the formation of a pharmaceutical composition, i.e., a dosage form capable of administration to the patient. One example of such a carrier is a pharmaceutically acceptable oil typically used for parenteral administration.

### Compounds

Compounds of the invention conform generally to Formula I:

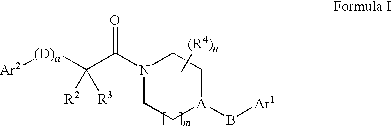

wherein:
Ar¹ is selected from the group consisting of aryl and heteroaryl, substituted with a CN group at the position alpha to the link with group B, further optionally-substituted with one or more substituents selected from the group consisting of alkyl, haloalkyl and halo;
Ar² is selected from the group consisting of aryl and heteroaryl, optionally substituted with one or more substituents selected from the group consisting of halo, alkyl, haloalkyl, alkoxy, haloalkoxy, NR⁵R⁶, O-alkylene-aryl, O-alkylene-heteroaryl, O-alkylene-O-alkyl, O-cycloalkyl, O-heterocycloalkyl, wherein any cyclic group may be further substituted with one or more substituents selected from the group consisting of alkyl and halo;
A is selected from the group consisting of C and N;
B is a bond when A is N and is group NR when A is C;
D is selected from the group consisting of NR¹ and O;
a is selected from the group consisting of 0 and 1;
m is selected from the group consisting of 1 and 2;
n is selected from the group consisting of 1, 2, 3 and 4;
R, R², R³, R⁵ and R⁶ are independently selected from the group consisting of H and alkyl;
R¹ is selected from the group consisting of H, alkyl, COR, CO₂R and SO₂R; and
R⁴ is selected from the group consisting of H, halo, CN, alkyl, haloalkyl, CH₂OR and CO₂R.

In a particular embodiment A is N and B is a bond.

In other embodiments m is 1; in yet others it is 2.

In yet other embodiments a is 0; in others it is 1.

In particular embodiments Ar¹ is a pyridyl group; in others it is a pyrazinyl group; in still others it is a phenyl group.

In other embodiments Ar² is a phenyl group; in others it is a pyridyl group.

It will be understood by those of skill in the art that when compounds of the present invention contain one or more chiral centers, the compounds of the invention may exist in, and be isolated as, enantiomeric or diastereomeric forms, or as a racemic mixture. The present invention includes any possible enantiomers, diastereomers, racemates or mixtures thereof, of a compound of Formula I. The optically active forms of the compound of the invention may be prepared, for example, by chiral chromatographic separation of a racemate or chemical or enzymatic resolution methodology, by synthesis from optically active starting materials or by asymmetric synthesis based on the procedures described thereafter.

It will also be understood by those of skill in the art that certain compounds of the present invention may exist in solvated, for example hydrated, as well as unsolvated forms. It will further be understood that the present invention encompasses all such solvated forms of the compounds of Formula I.

Within the scope of the invention are also salts of the compounds of Formula I. Generally, pharmaceutically acceptable salts of compounds of the present invention are obtained using standard procedures well known in the art, for example, by reacting a sufficiently basic compound, for example an alkyl amine with a suitable acid, for example, HCl or acetic acid, to afford a salt with a physiologically acceptable anion. It is also possible to make a corresponding alkali metal (such as sodium, potassium, or lithium) or an alkaline earth metal (such as a calcium) salt by treating a compound of the present invention having a suitably acidic proton, such as a carboxylic acid or a phenol, with one equivalent of an alkali metal or alkaline earth metal hydroxide or alkoxide (such as the ethoxide or methoxide), or a suitably basic organic amine (such as choline or meglumine) in an aqueous medium, followed by conventional purification techniques. Additionally, quaternary ammonium salts can be prepared by the addition of alkylating agents, for example, to neutral amines.

In one embodiment of the present invention, the compound of Formula I may be converted to a pharmaceutically acceptable salt or solvate thereof, particularly, an acid addition salt such as a hydrochloride, hydrobromide, phosphate, acetate, fumarate, maleate, tartrate, citrate, methanesulphonate or p-toluenesulphonate.

Specific examples of the present invention include the compounds 1 to 49, as illustrated in the following table, their pharmaceutically acceptable salts, hydrates, solvates, optical isomers, and combinations thereof:

| Compound | | |
|---|---|---|
| No. | STRUCTURE | NAME |
| 1 | 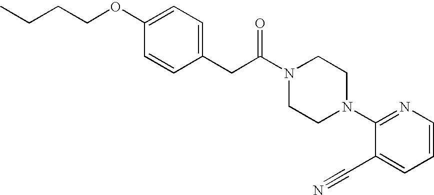 | 2-{4-[2-(4-Butoxy-phenyl)- acetyl]-piperazin-l-yl}- nicotinonitrile |
| 2 | 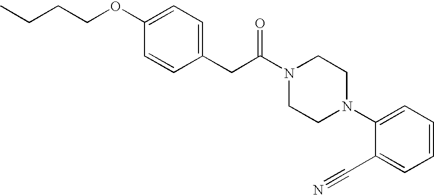 | 2-{4-[2-(4-Butoxy-phenyl)- acetyl]-piperazin-1-yl}- benzonitrile |
| 3 | 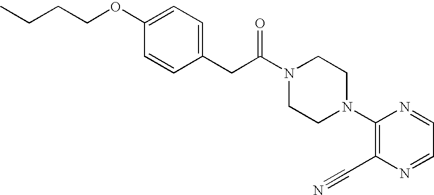 | 4-[2-(4-Butoxy-phenyl)-acetyl]- 3,4,5,6-tetrahydro-2H- [1,2′]bipyrazinyl-3′-carbonitrile |
| 4 | 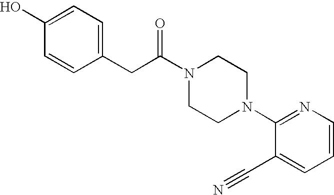 | 2-{4-[2-(4-Hydroxy-phenyl)- acetyl]-piperazin-1-yl}- nicotinonitrile |
| 5 | 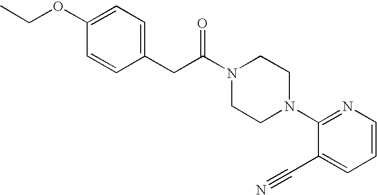 | 2-{4-[2-(4-Ethoxy-phenyl)- acetyl]-piperazin-1-yl}- nicotinonitrile |
| 6 | 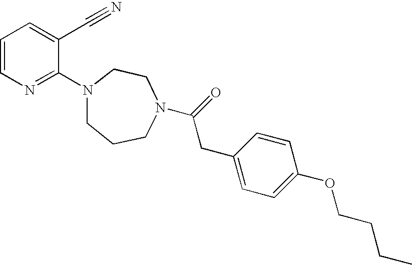 | 2-{4-[2-(4-Butoxy-phenyl)- acetyl]-[1,4]diazepan-1-yl}- nicotinonitrile |
| 7 | 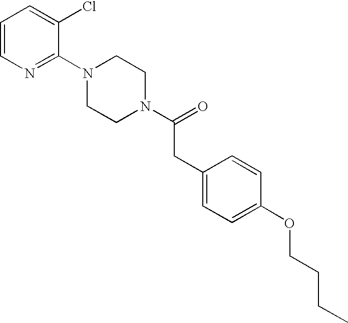 | 2-(4-Butoxy-phenyl)-1-[4-(3- chloro-pyridin-2-yl)-piperazin-1- yl]-ethanone |
| 8 | 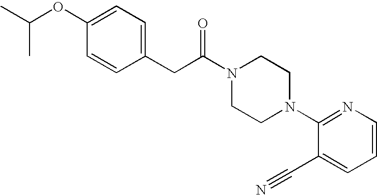 | 2-{4-[2-(4-lsopropoxy-phenyl)- acetyl]-piperazin-1-yl}- nicotinonitrile |
| 9 | 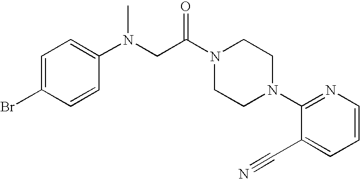 | 2-(4-{2-[(4-Bromo-phenyl)- methyl-amino]-acetyl)-piperazin- 1-yl)-nicotinonitrite |
| 10 | 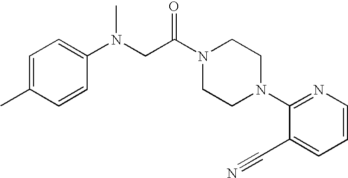 | 2-{4-[2-(Methyl-p-tolyl-amino)- acetyl]-piperazin-1-yl}- nicotinonitrile |
| 11 | 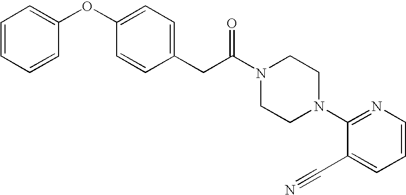 | 2-{4-[2-(4-Phenoxy-phenyl)- acetyl]-piperazin-1-yl}- nicotinonitrile |
| 12 | 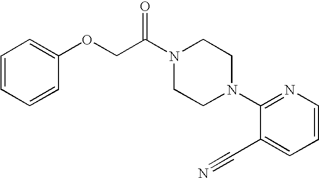 | 2-[4-(2-Phenoxy-acetyl)- piperazin-1-yl]-nicotinonitrile |
| 13 | 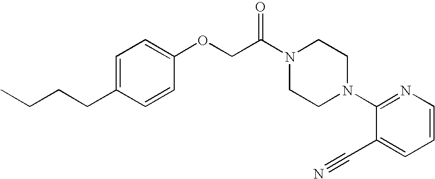 | 2-{4-[2-(4-Butyl-phenoxy)- acetyl]- piperazin-1-yl}-nicotinonitrile |
| 14 | 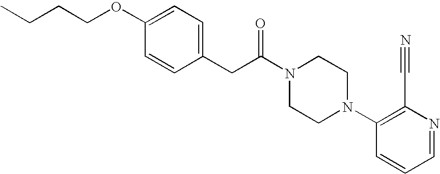 | 3-{4-[2-(4-Butoxy-phenyl)- acetyl]-piperazin-1-yl}-pyridine- 2-carbonitrile |
| 15 | 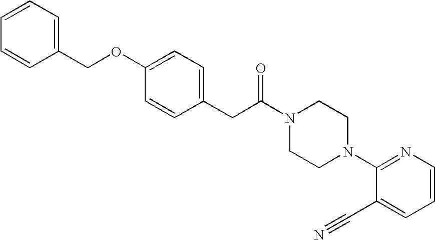 | 2-{4-[2-(4-Benzyloxy-phenyl)- acetyl]-piperazin-1-yl}- nicotinonitrile |
| 16 | 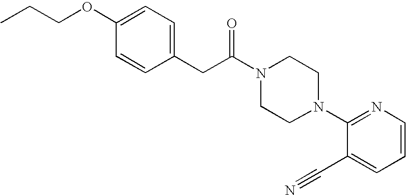 | 2-{4-[2-(4-Propoxy-phenyl)- acetyl]-piperazin-1-yl}- nicotinonitrile |
| 17 | 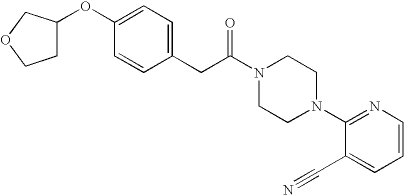 | 2-(4-{2-[4-(Tetrahydro-furan-3- yloxy)-phenyl]-acetyl}-piperazin-1-yl)- nicotinonitrile |
| 18 | 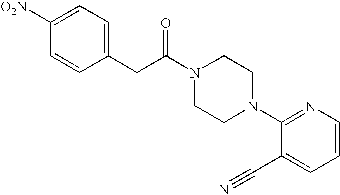 | 2-{4-[2-(4-Nitro-phenyl)-acetyl]- piperazin-1-yl}-nicotinonitrile |
| 19 | 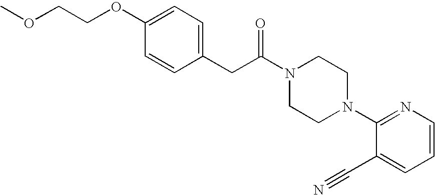 | 2-(4-{2-[4-(2-Methoxy-ethoxy)- phenyl]-acetyl}-piperazin-l-yl)- nicotinonitrile |
| 20 | 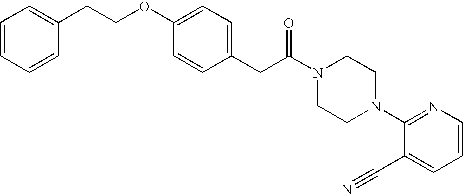 | 2-{4-[2-(4-Phenethyloxy-phenyl)- acetyl]-piperazin-1-yl}- nicotinonitrile |
| 21 | 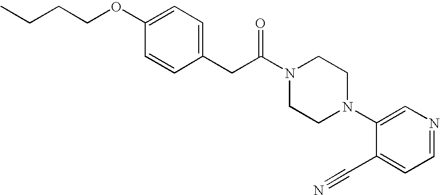 | 3-{4-[2-(4-Butoxy-phenyl)- acetyl]-piperazin-1-yl}- isonicotinonitrile |
| 22 | 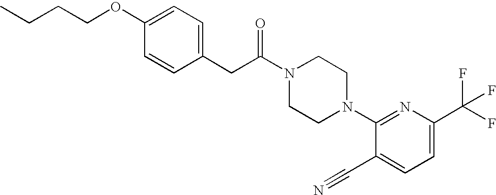 | 2-{4-[2-(4-Butoxy-phenyl)- acetyl]-piperazin-1-yl}-6- trifluoromethyl-nicotinonitrile |
| 23 | 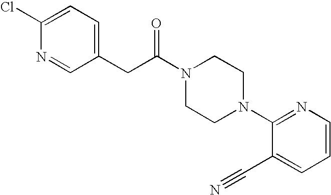 | 2-{4-[2-(6-Chloro-pyridin-3-yl)- acetyl]-piperazin-1-yl}- nicotinonitrile |
| 24 | 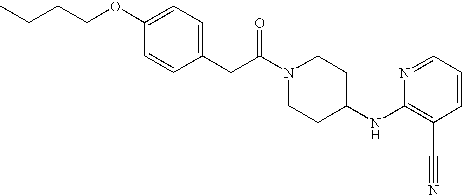 | 2-{1-[2-(4-Butoxy-phenyl)- acetyl]-piperidin-4-ylamino}- nicotinonitrile |
| 25 | 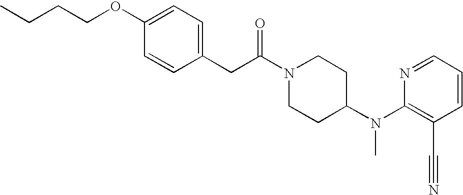 | 2-({1-[2-(4-Butoxy-phenyl)- acetyl]-piperidin-4-yl}-methyl- amino)-nicotinonitrile |
| 26 | 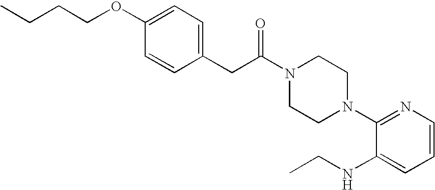 | 2-(4-Butoxy-phenyl)-1-[4-(3- ethylamino-pyridin-2-yl)- piperazin-1-yl]-ethanone |
| 27 | 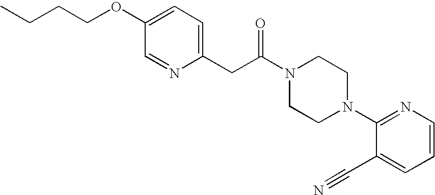 | 2-{4-[2-(5-Butoxy-pyridin-2-yl)- acetyl]-piperazin-1-yl}- nicotinonitrile |
| 28 | 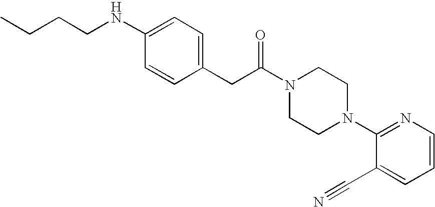 | 2-{4-[2-(4-Butylamino-phenyl)- acetyl]-piperazin-1-yl}- nicotinonitrile |
| 29 | 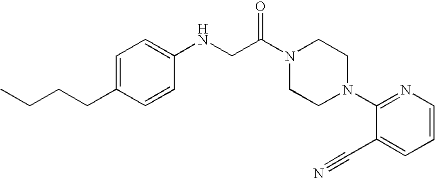 | 2-{4-[2-(4-Butyl-phenylamino)- acetyl]-piperazin-1-yl}- nicotinonitrile |
| 30 | 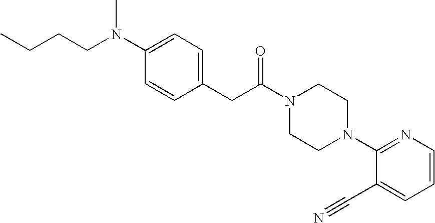 | 2-(4-{2-[4-(Butyl-methyl-amino)- phenyl]-acetyl}-piperazin-1-yl)- nicotinonitrile |
| 31 | 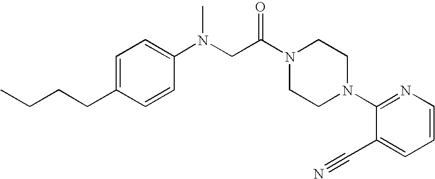 | 2-(4-{2-[(4-Butyl-phenyl)- methyl-amino]-acetyl}-piperazin- 1-yl)-nicotinonitrile |
| 32 | 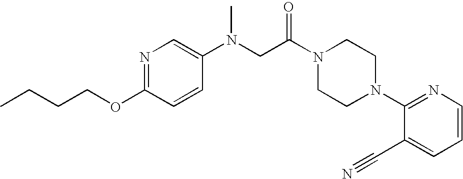 | 2-(4-{2-[(6-Butoxy-pyridin-3-yl)- methyl-amino]-acetyl}-piperazin- 1-yl)-nicotinonitrile |
| 33 | 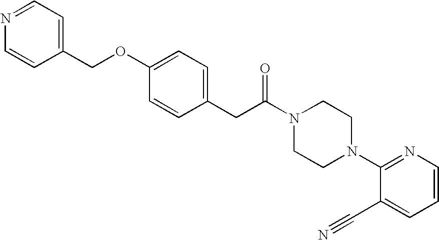 | 2-(4-{2-[4-(Pyridin-4- ylmethoxy)-phenyl]-acetyl}- piperazin-1-yl)-nicotinonitrile |
| 34 | 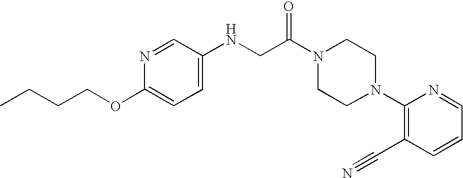 | 2-{4-[2-(6-Butoxy-pyridin-3- ylamino)-acetyl]-piperazin-1-yl}- nicotinonitrile |
| 35 | 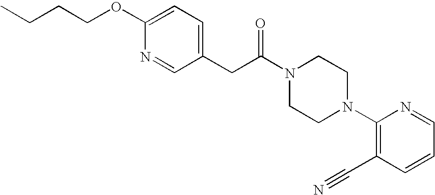 | 2-{4-[2-(6-Butoxy-pyridin-3-yl)- acetyl]-piperazin-1-yl}- nicotinonitrile |
| 36 | 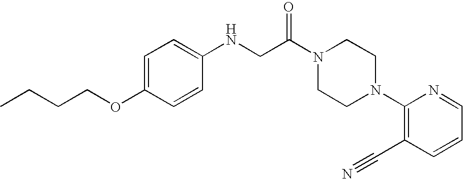 | 2-{4-[2-(4-Butoxy-phenylamino)- acetyl]-pperazin-1-yl}- nicotinonitrile |
| 37 | 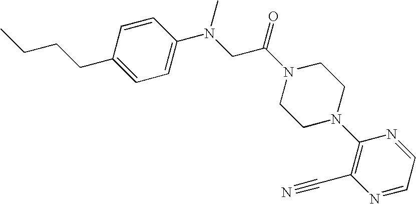 | 4-{2-[(4-Butyl-phenyl)-methyl- amino]-acetyl}-3,4,5,6- tetrahydro-2H-[1,2]bipyrazinyl- 3′-carbonitrile |
| 38 | 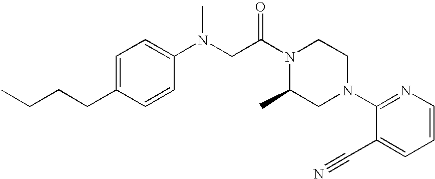 | 2-((R)-4-{2-[(4-Butyl-phenyl)- methyl-amino]-acetyl}-3-methyl- piperazin-1-yl)-nicotinonitrile |
| 39 | 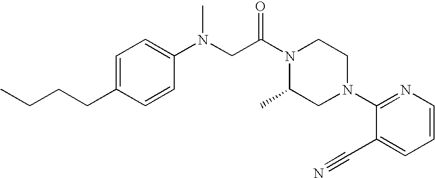 | 2-((S)-4-{2-[(4-Butyl-phenyl)- methyl-amino]-acetyl}-3-methyl- piperazin-1-yl)-nicotinonitrile |
| 40 | 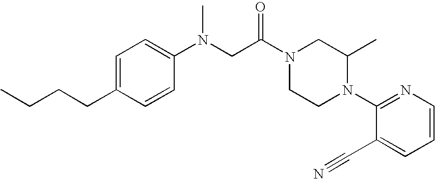 | 2-(4-{2-[(4-Butyl-phenyl)- methyl-amino]-acetyl}-2-methyl- piperazin-1-yl)-nicotinonitrile |
| 41 | 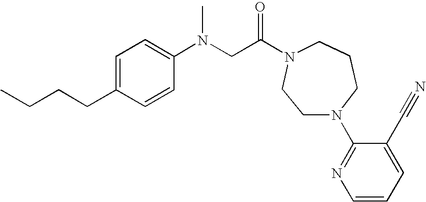 | 2-(4-{2-[(4-Butyl-phenyl)- methyl-amino]-acetyl}- [1,4]diazepan-1-yl)-nicotinonitrile |
| 42 | 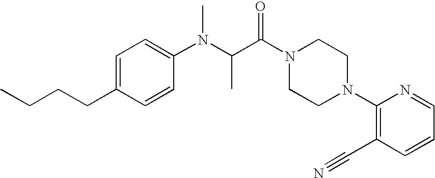 | 2-(4-{2-[(4-Butyl-phenyl)- methyl-amino]-propionyl}- piperazin-1-yl)-nicotinonitrile |
| 43 | 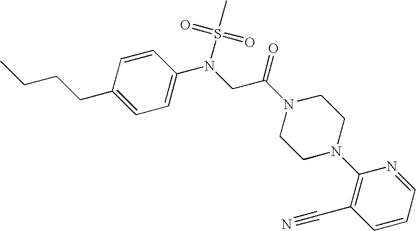 | N-(4-Butyl-phenyl)-N-{2-[4-(3- cyano-pyridin-2-yl)-piperazin-1- yl]-2-oxo-ethyl}- methanesulfonamide |
| 44 | 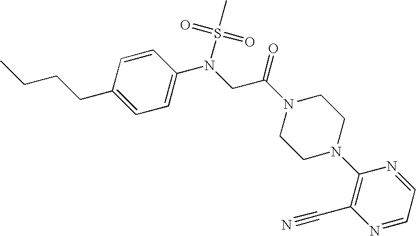 | N-(4-Butyl-phenyl)-N-{2-(3′- cyano-2,3,5,6-tetrahydro- [1,2]bipyrazinyl-4-yl)-2-oxo- ethyl]-methanesulfonamide |
| 45 | 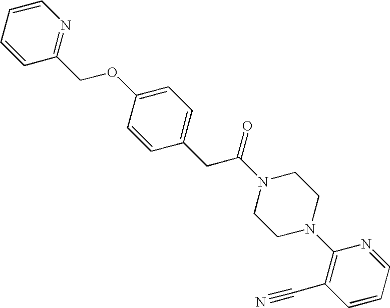 | 2-(4-{2-[4′-(Pyridin-2- ylmethoxy)-phenyl]-acetyl}- piperazin-1-yl)-nicotinonitrile |
| 46 | 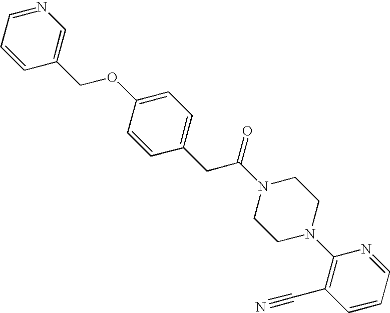 | 2-(4-{2-[4-(Pyridin-3- ylmethoxy)-phenyl]-acetyl)- piperazin-1-yl)-nicotinonitrile |
| 47 | 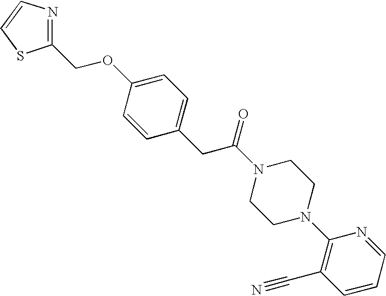 | 2-(4-{2-[4-(Thiazol-2- ylmethoxy)-phenyl]-acetyl}- piperazin-1-yl)-nicotinonitrile |
| 48 | 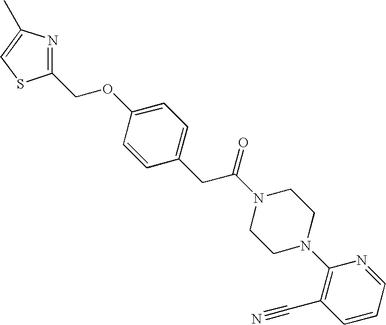 | 2-(4-{2-[4-(4-Methyl-thiazol-2- ylmethoxy)-phenyl]-acetyl}- piperazin-1-yl)-nicotinonitrile |
| 49 | 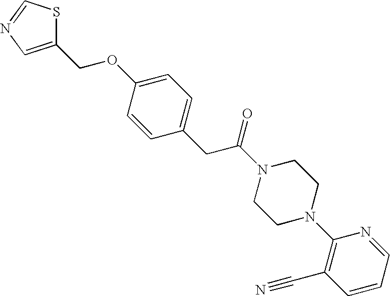 | 2-(4-{2-[4-(Thiazol-5- ylmethoxy)-phenyl]-acetyl}- piperazin-1-yl)-nicotinonitrile |

### Pharmaceutical Compositions

The compounds of the present invention may be formulated into conventional pharmaceutical compositions comprising a compound of Formula I, or a pharmaceutically acceptable salt or solvate thereof and a pharmaceutically acceptable carrier or excipient. The pharmaceutically acceptable carriers can be either solid or liquid. Solid form preparations include, but are not limited to, powders, tablets, dispersible granules, capsules, cachets, and suppositories.

A solid carrier can be one or more substances, which may also act as diluents, flavoring agents, solubilizers, lubricants, suspending agents, binders, or tablet disintegrating agents. A solid carrier can also be an encapsulating material.

In powders, the carrier is a finely divided solid, which is in a mixture with the finely divided compound of the invention, or the active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture is then poured into convenient sized moulds and allowed to cool and solidify.

Suitable carriers include, but are not limited to, magnesium carbonate, magnesium stearate, tale, lactose, sugar, pectin, dextrin, starch, tragacanth, methyl cellulose, sodium carboxymethyl cellulose, low-melting wax, cocoa butter, and the like.

The term composition is also intended to include the formulation of the active component with encapsulating material as a carrier providing a capsule in which the active component (with or without other carriers) is surrounded by a carrier which is thus in association with it. Similarly, cachets are included.

Tablets, powders, cachets, and capsules can be used as solid dosage forms suitable for oral administration.

Liquid form compositions include solutions, suspensions, and emulsions. For example, sterile water or water propylene glycol solutions of the active compounds may be liquid preparations suitable for parenteral administration. Liquid compositions can also be formulated in solution in aqueous polyethylene glycol solution.

Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art. Exemplary compositions intended for oral use may contain one or more coloring, sweetening, flavoring and/or preservative agents.

Depending on the mode of administration, the pharmaceutical composition will include from about 0.05% w (percent by weight) to about 99% w, more particularly, from about 0.10% w to 50% w, of the compound of the invention, all percentages by weight being based on the total weight of the composition.

A therapeutically effective amount for the practice of the present invention can be determined by one of ordinary skill in the art using known criteria including the age, weight and response of the individual patient, and interpreted within the context of the disease which is being treated or which is being prevented.

Medical Use

It has been found that the compounds according to the present invention selectively potentiate mGluR5 receptor function. Accordingly, the compounds of the present invention are expected to be useful in the treatment of conditions associated with inhibition of mGluR5 or conditions in which downstream pathways are altered by activation of mGluR5.

The Group I mGluR receptors including mGluR5 are highly expressed in the central and peripheral nervous system and in other tissues. Thus, it is expected that the compounds of the invention are well suited for the treatment of mGluR5-mediated disorders such as acute and chronic neurological and psychiatric disorders, gastrointestinal disorders, and chronic and acute pain disorders.

The invention relates to compounds of Formula I, as defined herein, for use in therapy.

The invention relates to compounds of Formula I, as defined herein, for use in treatment of mGluR5-mediated disorders.

One embodiment of the invention relates to the use of a Formula I compound for the manufacture of a medicament for the treatment of schizophrenia.

Another embodiment of the invention relates to the use of a Formula I compound for the manufacture of a medicament for the treatment of cognition.

The invention also provides a method of treatment of mGluR5-mediated disorders and any disorder listed above, in a patient suffering from, or at risk of, said condition, which comprises administering to the patient an effective amount of a compound of Formula I, as hereinbefore defined.

The dose required for the therapeutic or preventive treatment of a particular disorder will necessarily be varied depending on the host treated, the route of administration and the severity of the illness being treated.

In the context of the present specification, the term “therapy” and “treatment” includes prevention or prophylaxis, unless there are specific indications to the contrary. The terms “therapeutic” and “therapeutically” should be construed accordingly.

The term “disorder”, unless stated otherwise, means any condition and disease associated with metabotropic glutamate receptor activity.

### Non-Medical Use

In addition to their use in therapeutic medicine, the compounds of Formula I, as well as salts and hydrates of such compounds, are useful as pharmacological tools in the development and standardization of in vitro and in vivo test systems for the evaluation of the effects of inhibitors of mGluR related activity in laboratory animals such as cats, dogs, rabbits, monkeys, rats and mice, as part of the search for new therapeutics agents.

### Process for Preparation

Another aspect of the present invention provides processes for preparing compounds of Formula I, or salts or hydrates thereof. Processes for the preparation of the compounds in the present invention are described below.

Throughout the following description of such processes it is to be understood that, where appropriate, suitable protecting groups will be added to, and subsequently removed from, the various reactants and intermediates in a manner that will be readily understood by one skilled in the art of organic synthesis. Conventional procedures for using such protecting groups as well as examples of suitable protecting groups are described, for example, in “Protective Groups in Organic Synthesis”, T. W. Green, P. G. M. Wuts, Wiley-Interscience, New York, (1999). It also is to be understood that a transformation of a group or substituent into another group or substituent by chemical manipulation can be conducted on any intermediate or final product on the synthetic path toward the final product, in which the possible type of transformation is limited only by inherent incompatibility of other functionalities carried by the molecule at that stage to the conditions or reagents employed in the transformation. Such inherent incompatibilities, and ways to circumvent them by carrying out appropriate transformations and synthetic steps in a suitable order, will be readily understood to the one skilled in the art of organic synthesis. Examples of transformations are given below, and it is to be understood that the described transformations are not limited only to the generic groups or substituents for which the transformations are exemplified. References and descriptions on other suitable transformations are given in “Comprehensive Organic Transformations—A Guide to Functional Group Preparations” R. C. Larock, VHC Publishers, Inc, (1989). References and descriptions of other suitable reactions are described in textbooks of organic chemistry, for example, “Advanced Organic Chemistry”, March, 4th ed. McGraw Hill (1992) or, “Organic Synthesis”, Smith, McGraw Hill, (1994). Techniques for purification of intermediates and final products include for example, normal and reversed phase chromatography on column or rotating plate, recrystallization, distillation and liquid-liquid or solid-liquid extraction, which will be readily understood by the one skilled in the art. The definitions of substituents and groups are as in Formula I except where defined differently. The term “room temperature” and “ambient temperature” shall mean, unless otherwise specified, a temperature between 16 and 25° C.

Compounds of Formula I may be prepared according to methods shown in Schemes 1-5, below. It will be readily understood by those of skill in the art that the choice of route for a specific compound of the invention will be influenced by a number of factors including, but not limited to, availability of starting materials, the nature of any substituents etc. Unless otherwise indicated, the variables described in the following schemes have the same definitions as those given for Formula I, above.

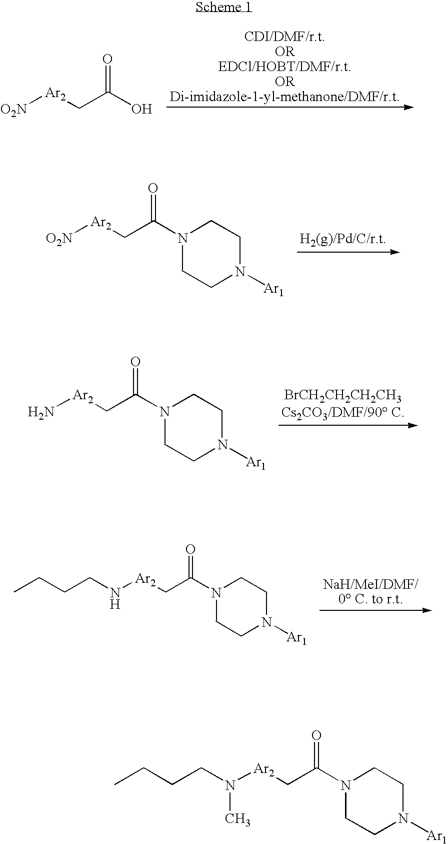

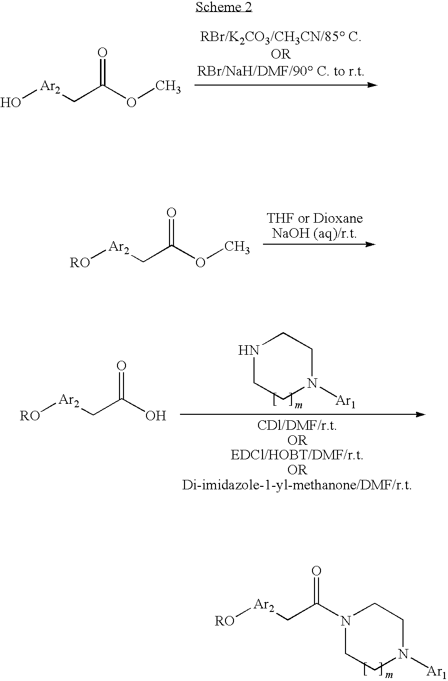

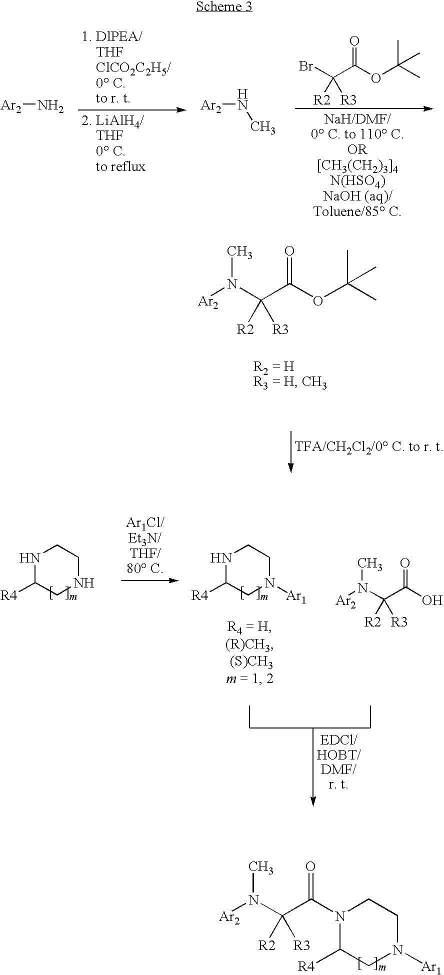

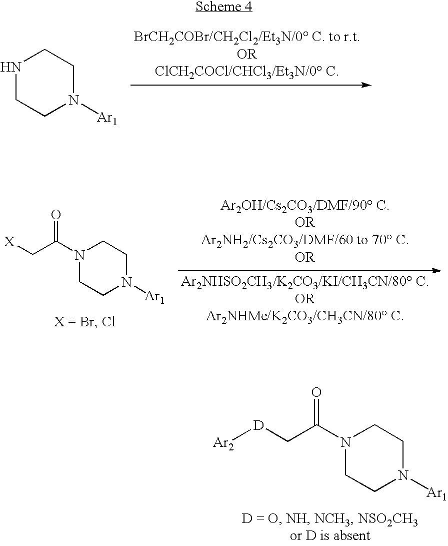

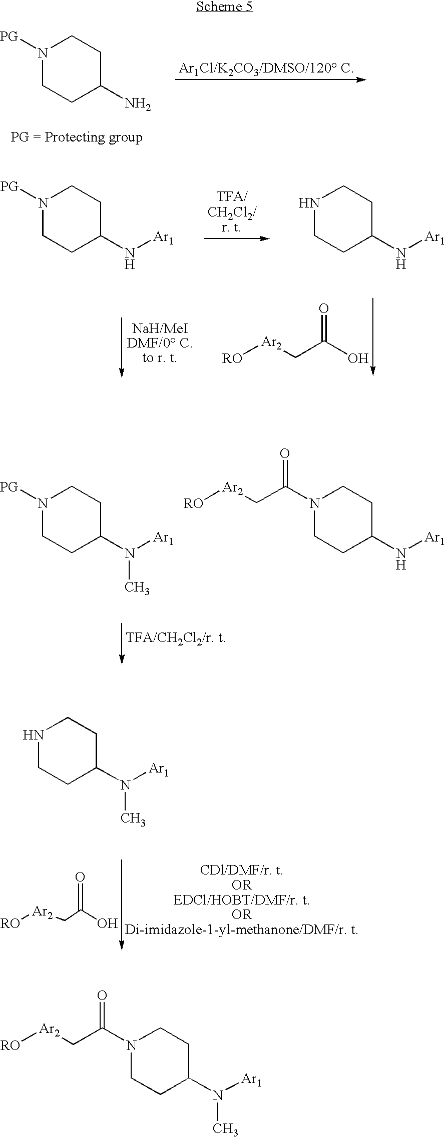

The invention is further illustrated by way of the following examples, which are intended to elaborate several embodiments of the invention. These examples are not intended to, nor are they to be construed to, limit the scope of the invention. It will be clear that the invention may be practiced otherwise than as particularly described herein. Numerous modifications and variations of the present invention are possible in view of the teachings herein and, therefore, are within the scope of the invention.

### EXAMPLES

### General Methods

All starting materials are commercially available or earlier described in the literature.

¹H and ¹³C NMR spectra were recorded either on Bruker 300, Bruker DPX400 or Varian +400 spectrometers operating at 300, 400 and 400 MHz for ¹H NMR respectively, using TMS or the residual solvent signal as reference, in deuterated chloroform as solvent unless otherwise indicated. All reported chemical shifts are in ppm on the delta-scale, and the fine splitting of the signals as appearing in the recordings (s: singlet, br s: broad singlet, d: doublet, t: triplet, q: quartet, in: multiplet). Unless otherwise indicated, in the tables below ¹H NMR data was obtained at 300 MHz, using CDCl₃ as the solvent.

Purification of products were also done using Chem Elut Extraction Columns (Varian, cat #1219-8002), Mega BE-SI (Bond Elut Silica) SPE Columns (Varian, cat #12256018; 12256026; 12256034), or by flash chromatography in silica-filled glass columns.

Microwave heating was performed in an Emrys Optimizer from Biotage/Personal Chemistry or a Smith Synthesizer Single-mode microwave cavity producing continuous irradiation at 2450 MHz (Personal Chemistry AB, Uppsala, Sweden).

### Pharmacological Assays

The pharmacological properties of the compounds of the invention can be analyzed using standard assays for functional activity. Examples of glutamate receptor assays are well known in the art as described in for example Aramori et al., Neuron 8:757 (1992), Tanabe et al., Neuron 8:169 (1992), Miller et al., J. Neuroscience 15: 6103 (1995), Balazs, et al., J. Neurochemistry 69:151 (1997). The methodology described in these publications is incorporated herein by reference. Conveniently, the compounds of the invention can be studied by means of an assay that measures the mobilization of intracellular calcium, [Ca²⁺]ᵢ in cells expressing mGluR5.

Intracellular calcium mobilization was measured by detecting changes in fluorescence of cells loaded with the fluorescent indicator fluo-3. Fluorescent signals were measured using the FLIPR system (Molecular Devices). A two addition experiment was used that could detect compounds that either activate or antagonize the receptor.

For FLIPR analysis, cells expressing human mGluR5d were seeded on collagen coated clear bottom 96-well plates with black sides and analysis of [Ca²⁺]ᵢ mobilization was done 24 hours after seeding.

FLIPR experiments were performed using a laser setting of 0.800 W and a 0.4 second CCD camera shutter speed. Each FLIPR experiment was initiated with 160 μL of buffer present in each well of the cell plate. After each addition of the compound, the fluorescence signal was sampled 50 times at 1 second intervals followed by 3 samples at 5 second intervals. Responses were measured as the peak height of the response within the sample period.

EC₅₀ and IC₅₀ determinations were made from data obtained from 8-point concentration response curves (CRC) performed in duplicate. Agonist CRC were generated by scaling all responses to the maximal response observed for the plate. Antagonist block of the agonist challenge was normalized to the average response of the agonist challenge in 14 control wells on the same plate.

We have validated a secondary functional assay for mGluR5d based on Inositol Phosphate (IP₃) turnover. IP₃ accumulation is measured as an index of receptor mediated phospholipase C turnover. GHEK cells stably expressing the human mGluR5d receptors were incubated with [3H] myo-inositol overnight, washed three times in HEPES buffered saline and pre-incubated for 10 minutes with 10 mM LiCl. Compounds (agonists) were added and incubated for 30 minutes at 37° C. Antagonist and potentiator activity was determined by pre-incubating test compounds for 15 minutes, then incubating in the presence of glutamate or DHPG (EC80 for antagonists, EC30 for potentiators) for 30 minutes. Reactions were terminated by the addition of perchloric acid (5%). Samples were collected and neutralized, and inositol phosphates were separated using Gravity-Fed Ion-Exchange Columns.

Generally, the compounds of the present invention were active in assays described herein at concentrations (or with EC₅₀ values) less than 10 μM. For example, compounds 1, 12, 16, 44 and 45 have EC₅₀ values of 5.1, 3.5, 2.8, 5.6 and 3.4 μM, respectively.

### Abbreviations

FLIPR Fluorometric Imaging Plate reader

### CCD Charge Coupled Device

### CRC Concentration Response Curve

GHEK Human Embryonic Kidney expressing Glutamate Transporter
HEPES 4-(2-hydroxyethyl)-1-piperazineethanesulfonic acid (buffer)
IP₃ inositol triphosphate
DHPG 3,5-dihydroxyphenylglycine;

### Example 1.1

### 2-{4-[2-(4-Butoxy-phenyl)-acetyl]-piperazin-1-yl}-nicotinonitrile

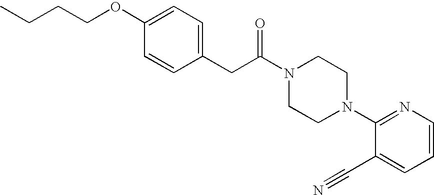

A mixture of 4-n-butoxyphenylacetic acid (121.7 mg, 0.58 mmol), 1-[2-(3-cyanopyridyl)]-piperazine (100 mg, 0.53 mmol), 1-ethyl-3-(3-dimethylaminopropyl)carbodiimide hydrochloride (112 mg, 0.58 mmol), 1-hydroxybenzotriazole hydrate (79 mg, 0.58 mmol) and N,N-dimethylformamide (2 mL) was stirred at room temperature overnight. The reaction mixture was diluted with ethyl acetate (10 mL) and sequentially washed with water (10 mL), saturated aqueous sodium bicarbonate (10 mL), water (10 mL) and brine (10 mL). The organic layer was dried over sodium sulfate, filtered and concentrated. The residue was purified on silica gel using hexanes:ethyl acetate (90:10 to 30:70) to give 2-{4-[2-(4-butoxy-phenyl)-acetyl]-piperazin-1-yl}-nicotinonitrile (188.4 mg, 94%) as a yellow solid.

In a similar manner the following compounds were synthesized:

| Example | Structure | Name | Yield |
|---|---|---|---|
| 1.2 | 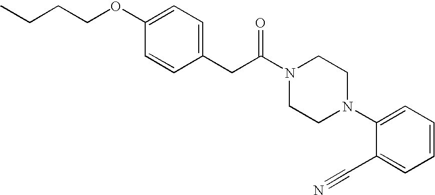 | 2-{4-[2-(4-Butoxy- phenyl)-acetyl]-piperazin- 1-yl}-benzonitrile | Yellow oil, 131.7 mg, 66% |
| 1.3 | 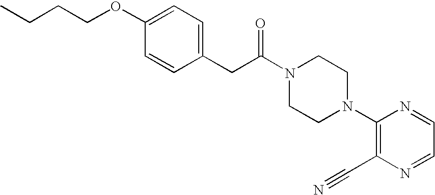 | 4-[2-(4-Butoxy-phenyl)- acetyl]-3,4,5,6-tetrahydro- 2H-[1,2]bipyrazinyl-3′ carbonitrile | Yellow solid, 94.9 mg, 96% |
| NMR | ¹H NMR (300 MHz, CDCl₃): δ(ppm) 8.28 (d, 1H), 8.08 (d, 1H), 7.18 (dd, | | |
| | 2H), 6.88 (dd, 2H), 3.96 (t, 2H), 3.83 (m, 2H), 3.76 (m, 4H), 3.62 (m, | | |
| | 4H), 1.77 (m, 2H), 1.50 (m, 2H), 0.99 (t, 3H). | | |
| 1.4 | 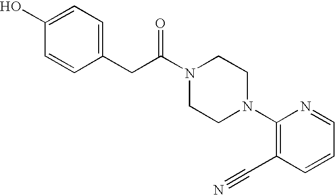 | 2-{4-[2-(4-Hydroxy- phenyl)-acetyl]-piperazin- 1-yl}-nicotinonitrile | Off-white solid, 49.7 mg, 48% |
| NMR | ¹H NMR (300 MHz, CD₃OD): δ(ppm) 8.38 (dd, 1H), 7.96 (dd, lH), 7.11 | | |
| | (dd, 2H), 6.94 (m, 1H), 6.77 (dd, 2H), 3.75 (m, 6H), 3.64 (m, 2H), 3.51 | | |
| | (m, 2H). | | |
| 1.5 | 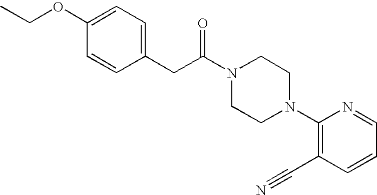 | 2-{4-[2-(4-Ethoxy- phenyl)-acetyl]-piperazin- 1-yl}-nicotinonitrile | Pale yellow oil, 64.1 mg, 57% |
| NMR | ¹H NMR (300 MHz, CDCl₃): δ(ppm) 8.36 (dd, 1H), 7.80 (dd, 1H), 7.18 | | |
| | (dd, 2H), 6.86 (m, 3H), 4.03 (q, 2H), 3.81 (m, 2H), 3.73 (s, 2H), 3.64 (m, | | |
| | 4H), 3.52 (m, 2H), 1.42 (t, 3H). | | |
| 1.6 | 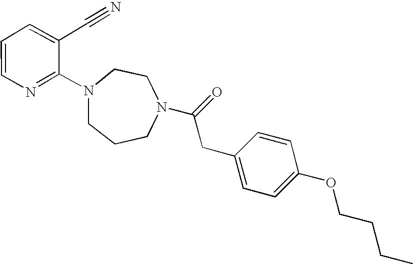 | 2-{4-[2-(4-Butoxy- phenyl)-acetyl]- [1,4]diazepan-1-yl}- nicotinonitrile | Yellow oil, 170 mg, 77% |
| NMR | ¹H NMR (300 MHz, CDCl₃): δ(ppm) 8.20 (m, 1H), 7.64 (m, 1H), 7.07 | | |
| | (m, 2H), 6.75 (m, 2H), 6.59 (m, 1H), 3.87 (m, 6H), 3.66 (s, 2H), 3.61 (m, | | |
| | 2H), 3.48 (m, 2H), 1.99 (m, 1H), 1.82 (m, 1H), 1.72 (m, 2H), 1.46 (m, | | |
| | 2H), 0.94 (t, 3H). | | |
| 1.7 | 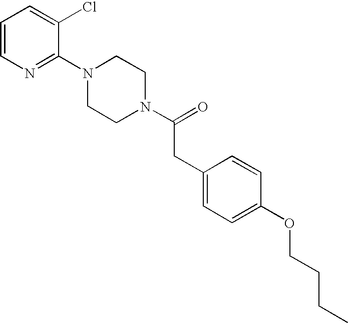 | 2-(4-Butoxy-phenyl)-1-[4- (3-chloro-pyridin-2-yl)- piperazin-1-yl]-ethanone | Clear oil, 166 mg, 76% |

| | |
|---|---|
| NMR | ¹H NMR(300 MHz, CDCl₃): δ(ppm) 8.11 (dd, 1H), 7.54 (dd, 1H), 7.14 |
| | (d, 2H), 6.80 (m, 3H), 3.89 (t, 2H), 3.76 (m, 2H), 3.68 (s, 2H), 3.56 (m, |
| | 2H), 3.26 (m, 2H), 3.13 (m, 2H), 1.72 (m, 2H), 1.46 (m, 2H), 0.93 (t, |
| | 3H). |

### Example 2.1

### 2-{4-[2-(4-Isopropoxy-phenyl)-acetyl]-piperazin-1-yl}-nicotinonitrile

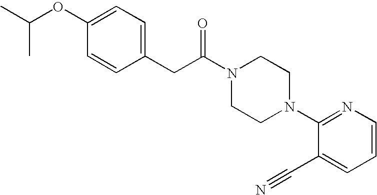

A suspension of 2-{4-[2-(4-hydroxy-phenyl)-acetyl]-piperazin-1-yl}-nicotinonitrile (36 mg, 0.11 mmol), potassium carbonate (46 mg, 0.33 mmol), 2-bromopropane (68.7 mg, 0.56 mmol) and acetonitrile (2 mL) was heated at 85° C. overnight. The reaction mixture was cooled to room temperature, filtered and concentrated. The residue was purified on silica gel using hexanes:ethyl acetate (70:30 to 30:70) to give 2-{4-[2-(4-isopropoxy-phenyl)-acetyl]-piperazin-1-yl}-nicotinonitrile (30.6 mg, 75%) as a pale yellow oil. ¹H NMR (300 MHz, CDCl₃): δ(ppm) 8.34 (dd, 1H), 7.79 (dd, 7.15 (dd, 2H), 6.83 (m, 3H), 4.52 (m, 1H), 3.80 (m, 2H), 3.71 (s, 2H), 3.63 (m, 4H), 3.51 (m, 2H), 1.32 (d, 6H).

### Example 3.1

### 2-[4-(2-Chloro-acetyl)-piperazin-1-yl]-nicotinonitrile

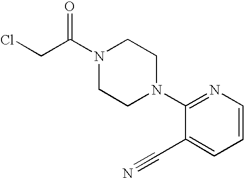

To a mixture of 2-piperazin-1-yl-nicotinonitrile (2.0 g, 10.63 mmol) and chloroform (25 mL) under nitrogen at 0° C., was added triethylamine (4.4 mL, 31.89 mmol). Chloroacetyl chloride (0.93 mL, 11.69 mmol) was then added dropwise and the reaction mixture was stirred at 0° C. for 3 hours. Water was added and the mixture was extracted with dichloromethane (3×). The combined organic layer was washed with water, washed with brine, dried over sodium sulfate, filtered and concentrated. The residue was purified on silica gel using hexanes:ethyl acetate (50:50) to give 2-[4-(2-chloro-acetyl)-piperazin-1-yl]-nicotinonitrile (1.43 g, 51%) as an off-white solid. ¹H NMR (300 MHz, CDCl₃): δ(ppm) 8.37 (dd, 1H), 7.82 (dd, 1H), 6.85 (m, 1H), 4.11 (s, 2H), 3.74 (m, 8H).

In a similar manner the following compounds were synthesized:

| Example | Structure | Name | Yield |
|---|---|---|---|
| 3.2 | 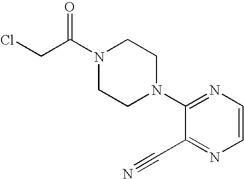 | 4-(2- Chloro- acetyl)- 3,4,5,6- tetra- hydro- 2H- [1,2′] bi- pyrazinyl- 3′- car- bonitrile | 0.66 g, 31% |

| | |
|---|---|
| NMR | ¹H NMR (300 MHz, CDCl₃): δ(ppm) 8.11 (d, 1H), 7.54 |
| | (d, 1H), 7.14 (s, 2H), 6.80 (m, 2H), 3.89 |
| | (m, 4H), 3.76 (m, 2H). |

### Example 4.1

### {2-[4-(3-Cyano-pyridin-2-yl)-piperazin-1-yl]-2-oxo-ethyl}-methyl-carbamic acid tert-butyl ester

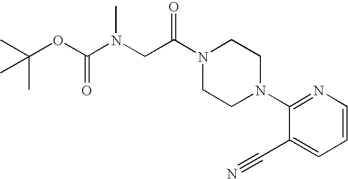

A mixture of (tert-butoxycarbonyl-methyl-amino)-acetic acid (1.00 g, 5.29 mmol), 2-(1-piperazinyl)-3-pyridine carbonitrile (1.09 g, 5.81 mmol), 1-ethyl-3-(3-dimethylaminopropyl)carbodiimide hydrochloride (1.11 g, 5.81 mmol), 1-hydroxybenzotriazole hydrate (0.78 g, 5.81 mmol) and N,N-dimethylformamide (10 mL) was stirred at room temperature overnight. The reaction mixture was diluted with ethyl acetate (100 mL) and sequentially washed with water (75 mL), saturated aqueous sodium bicarbonate (75 mL), water (75 mL) and brine (75 mL). The organic layer was dried over sodium sulfate, filtered and concentrated. The residue was purified on silica gel using hexanes:ethyl acetate (50:50 to 0:100) to give {2-[4-(3-cyano-pyridin-2-yl)-piperazin-1-yl]-2-oxo-ethyl}-methyl-carbamic acid tert-butyl ester (1.89 g, 99%) as a clear oil, ¹H NMR (300 MHz, CDCl₃): δ(ppm) 8.38 (m, 1H), 7.83 (dd, 1H), 6.84 (m, 1H), 4.12 (s, 2H), 3.72 (m, 8H), 2.96 (s, 3H), 1.49 (s, 9H).

### Example 5.1

### 2-[4-(2-Methylamino-acetyl)-piperazin-1-yl]-nicotinonitrile

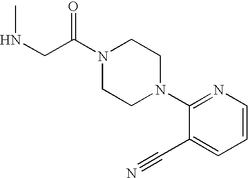

To a solution of {2-[4-(3-cyano-pyridin-2-yl)-piperazin-1-yl]-2-oxo-ethyl}-methyl-carbamic acid tert-butyl ester (1.89 g, 5.27 mmol) in dichloromethane (10 mL) at 0° C., was added trifluoroacetic acid (10 mL). The reaction mixture was stirred at room temperature for 1 hour. The reaction mixture was concentrated and to the residue was added saturated aqueous sodium bicarbonate (15 mL). The mixture was extracted with ethyl acetate (10×50 mL) and the combined organic layer was dried over sodium sulfate, filtered and concentrated. The residue was purified on silica gel using dichloromethane:methanol (100:0 to 80:20) to give 2-[4-(2-methylamino-acetyl)-piperazin-1-yl]-nicotinonitrile (1.56 g) as a yellow gel. ¹H NMR (300 MHz, acetone-d₆): δ(ppm) 8.43 (dd, 1H), 8.01 (dd, 1H), 6.97 (m, 1H), 4.42 (bs, 1H), 3.75 (m, 10H), 2.56 (s, 3H).

In a similar manner the following compounds were synthesized:

| Example | Structure | Name | Yield |
|---|---|---|---|
| 5.2 | 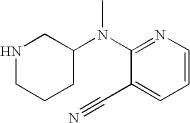 | 2-(Methyl- piperidin-4-yl- amino)- nicotinonitrile | 500 mg 56% |
| 5.3 | 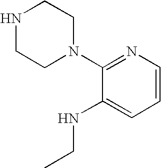 | Ethyl-(2- piperazin-1-yl- pyridin-3- yl)-amine | 1.3 g 96% |

### Example 6.1

### 2-(4-{2-[(4-Bromo-phenyl)-methyl-amino]-acetyl}-piperazin-1-yl)-nicotinonitrile

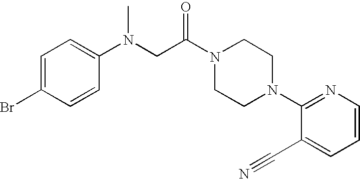

To a mixture of 2-[4-(2-methylamino-acetyl)-piperazin-1-yl]-nicotinonitrile (100.0 mg, 0.386 mmol) and tetrahydrofuran (6 mL) were added copper (II) acetate (113.4 mg, 0.772 mmol), 4-bromophenyl boronic acid (310.1 mg, 1.544 mmol), 4 Å molecular sieves (100 mg) and triethylamine (0.215 mL, 1.544 mmol). The reaction mixture was heated at 60° C. overnight. The reaction mixture was cooled to room temperature, filtered through Diatomaceous earth, water was added and the mixture was extracted with ethyl acetate (3×). The combined organic layer was washed with brine, dried over sodium sulfate, filtered and concentrated. The residue was purified on silica gel using hexanes:ethyl acetate (50:50 to 0:100) to give 2-(4-{2-[(4-bromo-phenyl)-methyl-amino]-acetyl}-piperazin-1-yl)-nicotinonitrile (36.0 mg, 23%) as a clear oil. ¹H NMR (300 MHz, CDCl₃): δ(ppm) 8.39 (dd, 1H), 7.83 (dd, 1H), 7.29 (d, 2H), 6.86 (m, 1H), 6.57 (d, 2H), 4.15 (s, 2H), 3.73 (m, 8H), 3.04 (s, 3H).

In a similar manner the following compounds were synthesized:

| Example | Structure | Name | Yield |
|---|---|---|---|
| 6.2 | 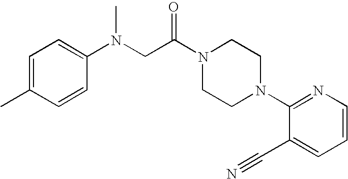 | 2-{4-[2-(Methyl-p-tolyl- amino)-acetyl]-piperazin- 1-yl}-nicotinonitrile | Pale brown oil, 32.8 mg, 24% |

| | |
|---|---|
| NMR | ¹H NMR (300 MHz, CDCl₃): δ(ppm) 8.38 (dd, 1H), 7.81 (dd, 1H), 7.06 |
| | (d, 2H), 6.84 (m, 1H), 6.68 (d, 2H), 4.11 (s, 2H), 3.78 (m, 2H), 3.70 (m, |
| | 6H), 3.02 (s, 3H), 2.25 (s, 3H). |

### Example 7.1

### 2-{4-[2-(4-Phenoxy-phenyl)-acetyl]-piperazin-1-yl}-nicotinonitrile

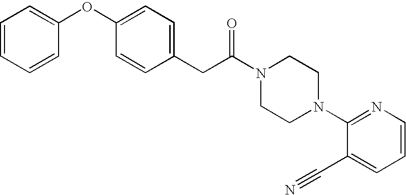

A mixture of (4-phenoxy-phenyl)-acetic acid (607 mg, 2.66 mmol), di-imidazol-1-yl-methanone (431 mg, 2.66 mmol) and N,N-dimethylformamide (30 mL) was stirred at room temperature for 2 hours under nitrogen. A solution of 2-piperazin-1-yl-nicotinonitrile (500 mg, 2.66 mmol) in N,N-dimethylformamide (3 mL) was then added. The reaction mixture was stirred for an additional 1.5 hours. The reaction mixture was diluted with ethyl acetate (10 mL) and sequentially washed with water (10 mL), saturated aqueous sodium bicarbonate (10 mL), water (10 mL) and brine (10 mL). The organic layer was dried over sodium sulfate, filtered and concentrated to give 2-{4-[2-(4-phenoxy-phenyl)-acetyl]-piperazin-1-yl}-nicotinonitrile (quantity; yield). ¹H NMR (300 MHz, CDCl₃): δ(ppm) 8.14 (dd, 1H), 7.58 (dd, 1H), 7.08 (m, 4H), 6.82 (m, 5H), 6.62 (m, 1H), 3.60 (m, 4H), 3.43 (m, 4H), 3.36 (m, 2H).

### Example 8.1

### 2-[4-(2-Bromo-acetyl)-piperazin-1-yl]-nicotinonitrile

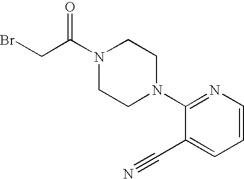

To a mixture of 2-piperazin-1-yl-nicotinonitrile (1.36 g, 7.23 mmol), bromo-acetyl bromide (0.63 mL, 7.23 mmol) and dichloromethane (41 mL) at 0° C., was added triethylamine (1.52 mL, 10.90 mmol). The reaction mixture was stirred at room temperature for 5 hours. Water was added and the mixture was extracted with dichloromethane (3×). The combined organic layer was washed with water, washed with brine, dried over sodium sulfate, filtered and concentrated to give 2-[4-(2-bromo-acetyl)-piperazin-1-yl]-nicotinonitrile (quantity; yield). The product was used without further purification.

### Example 9.1

### 2-[4-(2-Phenoxy-acetyl)-piperazin-1-yl]-nicotinonitrile

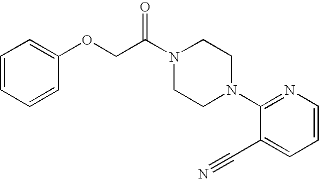

A mixture of 2-[4-(2-bromo-acetyl)-piperazin-1-yl]-nicotinonitrile (822 mg, 2.66 mmol), phenol (250 mg, 2.66 mmol), cesium carbonate (866 mg, 2.66 mmol) and N,N-dimethylformamide was stirred at 90° C. for 5 hours. The reaction mixture was cooled to room temperature, water was added and the mixture was extracted with ethyl acetate (3×). The combined organic layer was washed with water, washed with brine, dried over sodium sulfate, filtered and concentrated to give 2-[4-(2-phenoxy-acetyl)-piperazin-1-yl]-nicotinonitrile. ¹H NMR (300 MHz, CDCl₃): δ(ppm) 8.16 (dd, 1H), 7.60 (dd, 1H), 7.10 (m, 2H), 6.78 (m, 3H), 6.63 (m, 1H), 4.54 (s, 2H), 3.53 (m, 8H).

In a similar manner the following compounds were synthesized:

| Example | Structure | Name | Yield |
|---|---|---|---|
| 9.2 | 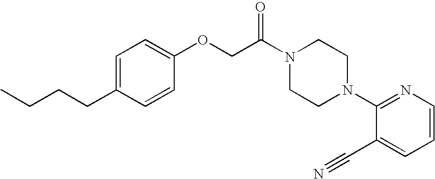 | 2-{4-[2-(4-Butyl- phenoxy)-acetyl]- piperazin-1-yl}- nicotinonitrile | |

| | |
|---|---|
| NMR | ¹H NMR (300 MHz, CDCl₃): δ(ppm) 8.15 (dd, 1H), 7.60 (dd, 1H), 6.85 |
| | (d, 2H), 6.65 (m, 3H), 4.50 (s, 2H), 3.55 (m, 8H), 2.35 (t, 2H), 1.35 (m, |
| | 2H), 1.10 (m, 2H), 0.75 (t, 3H). |

### Example 10.1

### (4-Propoxy-phenyl)-acetic acid methyl ester

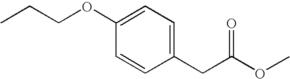

A mixture of (4-hydroxy-phenyl)-acetic acid methyl ester (3.00 g, 18.05 mmol), 1-iodopropane (1.76 mL, 18.05 mmol), cesium carbonate (8.82 g, 27.07 mmol) and N,N-dimethylformamide was stirred at 90° C. for 2 hours. The reaction mixture was cooled to room temperature, water was added and the mixture was extracted with ethyl acetate (3×). The combined organic layer was washed with water, washed with brine, dried over sodium sulfate, filtered and concentrated to give (4-propoxy-phenyl)-acetic acid methyl ester (quantity; yield). GC/MS m/z 398, 253, 210, 183, 148, 77.

### Example 11.1

### (4-Butoxy-phenyl)-acetic acid methyl ester

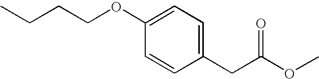

A mixture of (4-hydroxy-phenyl)-acetic acid methyl ester (3.0 g, 0.018 mol), 1-bromo-butane (3.88 mL, 0.036 mol), cesium carbonate (8.0 g, 0.027 mol) and N,N-dimethylformamide (40 mL) was stirred at 80-100° C. for 3 hours. The reaction mixture was cooled to room temperature, water was added and the mixture was extracted with ethyl acetate (3×). The combined organic layer was washed with water, washed with brine, dried over sodium sulfate, filtered and concentrated to give (4-butoxy-phenyl)-acetic acid methyl ester (4.0 g, 100%). The product was used without further purification.

### Example 12.1

### (4-Propoxy-phenyl)-acetic acid

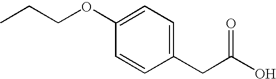

To a solution of (4-propoxy-phenyl)-acetic acid methyl ester (3.82 g, 18.34 mmol) in tetrahydrofuran (100 mL) was added a solution of sodium hydroxide (1.47 g, 36.75 mmol) in water. The reaction mixture was stirred at room temperature for 5 hours. The reaction mixture was acidified and extracted with ethyl acetate. The organic layer was washed with brine, dried over sodium sulfate, filtered and concentrated to give (4-propoxy-phenyl)-acetic acid. GC/MS m/z 194, 152, 107, 89, 77, 51, 45.

In a similar manner the following compounds were synthesized:

| Example | Structure | Name | Yield |
|---|---|---|---|
| 12.2 | 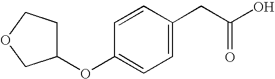 | [4-(Tetrahydro-furan-3- yloxy)-phenyl]-acetic acid | 4.68 g, 73% |
| 12.3 | 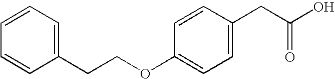 | (4-Phenethyloxy-phenyl)- acetic acid | |
| GC/MS | GC/MS m/z 256, 106, 91, 77. | | |
| 12.4 | 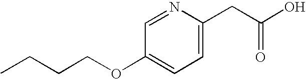 | (5-Butoxy-pyridin-2-yl)- acetic acid | |
| 12.5 | 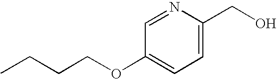 | (5-Butoxy-pyridin-2-yl)- methanol | 3.50 g, 92% |
| GC/MS | GC/MS m/z 181, 124, 96, 41. | | |
| 12.6 | 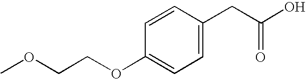 | [4-(2-Methoxy-ethoxy)- phenyl]-acetic acid | 4.65 g, 88% |

| | |
|---|---|
| GC/MS | GC/MS m/z 210, 152, 107, 77, 59. |

### Example 13.1

### (4-Butoxy-phenyl)-acetic acid

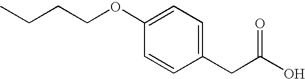

To a solution of (4-butoxy-phenyl)-acetic acid methyl ester (4.0 g, 0.018 mol) in dioxane (100 mL) was added a solution of sodium hydroxide (1.4 g, 0.036 mol) in water (20 mL). The reaction mixture was stirred at room temperature for 2 hours. The reaction mixture was acidified and extracted with ethyl acetate. The organic layer was washed with brine, dried over sodium sulfate, filtered and concentrated to give (4-butoxy-phenyl)-acetic acid. GC/MS m/z 208, 152, 107, 77.

### Example 14.1

### 3-Bromo-pyridine-2-carbonitrile

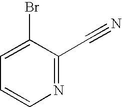

To a solution of 3-bromo-pyridine (5.0 g, 0.032 mol) in trifluoroacetic anhydride (25 mL) at 0° C., was slowly added nitric acid (4 mL, 0.064 mol). The reaction mixture was stiffed at room temperature for 1 hour and then cooled to 0° C. A solution of potassium cyanide (10.0 g, 0.16 mol) and sodium acetate (13.0 g, 0.16 mol) in water (50 mL) was added dropwise. The reaction mixture was stirred at room temperature for 12 hours and to it was then added dichloromethane and water. The organic layer was separated and the aqueous layer was further extracted with dichloromethane (3×). The combined organic layer was dried over sodium sulfate, filtered and concentrated. The residue was purified on silica gel to give 3-bromo-pyridine-2-carbonitrile (1.0 g, 17%).

### Example 15.1

### 3-piperazin-1-yl-pyridine-2-carbonitrile

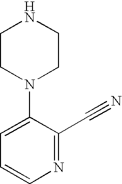

A mixture of 3-bromo-pyridine-2-carbonitrile (500 mg, 2.73 mmol), piperazine (2.3 g, 26.70 mmol) and methyl sulfoxide (20 mL) was heated at 150° C. for 15 minutes in a microwave. The reaction mixture was cooled to room temperature and water was added. The mixture was extracted with ethyl acetate and the organic layer was dried over sodium sulfate, filtered and concentrated. The residue was purified on silica gel to give 3-piperazin-1-yl-pyridine-2-carbonitrile. GC/MS m/z 188, 146, 104, 76, 56, 42.

### Example 16.1

### 3-{4-[2-(4-Butoxy-phenyl)-acetyl]-piperazin-1-yl}-pyridine-2-carbonitrile

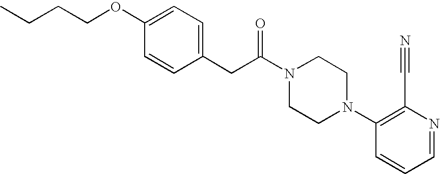

To a mixture of (4-butoxy-phenyl)-acetic acid (442 mg, 2.12 mmol) and N,N-dimethylformamide (25 mL) was added 1,1′-carbonyldiimidazole (345 mg, 2.12 mmol). The reaction mixture was stirred at room temperature for 30 minutes and then 3-piperazin-1-yl-pyridine-2-carbonitrile (400 mg, 2.12 mmol) was added. The reaction mixture was stirred at room temperature overnight. The reaction mixture was diluted with ethyl acetate and washed with water. The organic layer was separated, washed with brine, dried over sodium sulfate, filtered and concentrated. The residue was purified on silica gel using hexanes:ethyl acetate (70:30) to give 3-{4-[2-(4-butoxy-phenyl)-acetyl]-piperazin-1-yl}-pyridine-2-carbonitrile (250 mg, 31%). ¹H NMR (300 MHz, CDCl₃): δ(ppm) 8.10 (m, 1H), 7.20 (m, 1H), 7.10 (d, 1H), 6.95 (d, 2H), 6.63 (d, 2H), 3.70 (t, 2H), 3.63 (m, 2H), 3.50 (m, 4H), 2.92 (m, 2H), 2.83 (m, 2H), 1.50 (m, 2H), 1.27 (m, 2H), 0.70 (t, 3H).

In a similar manner the following compounds were synthesized;

| Example | Structure | Name | Yield |
|---|---|---|---|
| 16.2 | 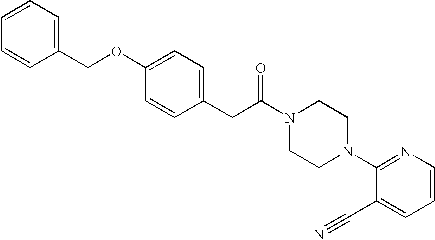 | 2-{4-[2-(4-Benzyloxy- phenyl)-acetyl]-piperazin- 1-yl}-nicotinonitrile | 350 mg, 32% |
| NMR | ¹H NMR (300 MHz, CDCl₃): δ(ppm) 8.12 (m, 1H), 7.54 (dd, 1H), 7.12 | | |
| | (m, 5H), 6.97 (d, 2H), 6.72 (dd, 2H), 6.56 (m, 1H), 4.81 (s, 2H), 3.42 (m, | | |
| | 10H). | | |
| 16.3 | 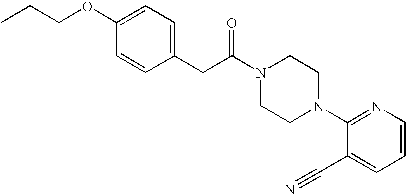 | 2-{4-[2-(4-Propoxy- phenyl)-acetyl]-piperazin- 1-yl}-nicotinonitrile | |
| NMR | ¹H NMR (300 MHz, CDCl₃): δ(ppm) 8.13 (m, 1H), 7.57 (dd, 1H), 6.96 | | |
| | (d, 2H), 6.61 (m, 3H), 3.45 (m, 12H), 1.58 (m, 2H), 0.82 (t, 3H). | | |
| 16.4 | 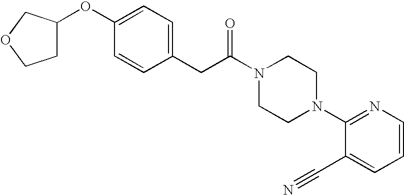 | 2-(4-{2-[4-(Tetrahydro- furan-3-yloxy)-phenyl]- acetyl-piperazin-1-yl) nicotinonitrile | 100 mg, 11% |
| NMR | ¹H NMR (300 MHz, CDCl₃): δ(ppm) 8.15 (dd, 1H), 7.58 (dd, 1H), 6.96 | | |
| | (d, 2H), 6.62 (m, 3H), 4.70 (m, 1H), 3.55 (m, 14H), 1.98 (m, 2H). | | |
| 16.5 | 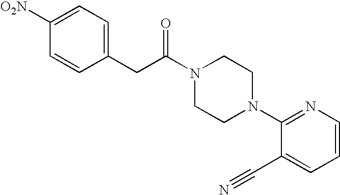 | 2-{4-[2-(4-Nitro-phenyl)- acetyl]-piperazin-1-yl}- nicotinonitrile | 1.28 g, 66% |
| GC/MS | GC/MS m/z 351, 158, 145, 132, 56. | | |
| 16.6 | 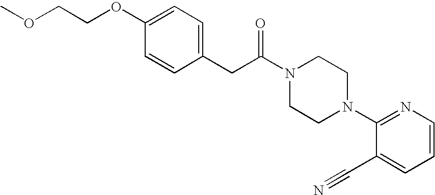 | 2-(4-{2-[4-(2-Methoxy- ethoxy)-phenyl]-acetyl}- piperazin-1-yl)- nicotinonitrile | 148 mg, 10% |
| NMR | ¹H NMR (300 MHz, CDCl₃): δ(ppm) 8.14 (dd, 1H), 7.58 (dd, 1H), 6.97 | | |
| | (d, 2H), 6.70 (d, 2H), 6.62 (m, 1H), 3.89 (m, 2H), 3.43 (m, 15H). | | |
| 16.7 | 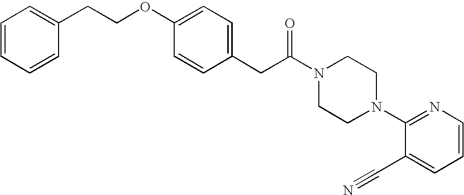 | 2-{4-[2-(4-Phenethyloxy- phenyl)-acetyl]-piperazin- 1-yl}-nicotinonitrile | 125 mg, 12% |
| NMR | ¹H NMR (300 MHz, CDCl₃): δ(ppm) 8.13 (dd, 1H), 7.56 (dd, 1H), 7.05 | | |
| | (m, 5H), 6.97 (d, 2H), 6.66 (d, 2H), 6.59 (m, 1H), 3.95 (m, 2H), 3.44 (m, | | |
| | 10H), 2.90 (m, 2H). | | |
| 16.8 | 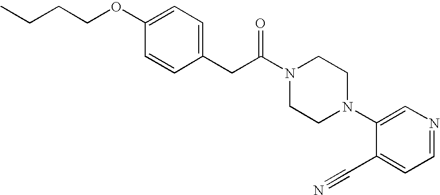 | 3-{4-[2-(4-Butoxy- phenyl)-acetyl]-piperazin- 1-yl}-isonicotinonitrile | 320 mg, 35% |
| NMR | ¹H NMR (300 MHz, CDCl₃): δ(ppm) 8.15 (s, 1H), 8.11 (d, 1H), 7.19 (d, | | |
| | 1H), 6.95 (d, 2H), 6.65 (d, 2H), 3.72 (t, 2H), 3.64 (m, 2H), 3.51 (s, 2H), | | |
| | 3.45 (m, 2H), 3.03 (m, 2H), 2.89 (m, 2H), 1.54 (m, 2H), 1.27 (m, 2H), | | |
| | 0.75 (t, 3H). | | |
| 16.9 | 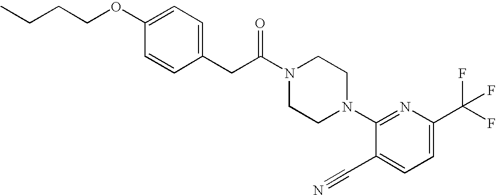 | 2-{4-[2-(4-Butoxy- phenyl)-acetyl]-piperazin- 1-yl}-6-trifluoromethyl- nicotinonitrile | 410 mg, 51% |
| NMR | ¹H NMR (300 MHz, CDCl₃): δ(ppm) 7.75 (d, 1H), 7.04 (d, 2H), 6.94 (d, | | |
| | 1H), 6.68 (d, 2H), 3.76 (t, 2H), 3.61 (m, 4H), 3.53 (s, 2H), 3.44 (m, 4H), | | |
| | 1.57 (m, 2H), 1.30 (m, 2H), 0.78 (t, 3H). | | |
| 16.10 | 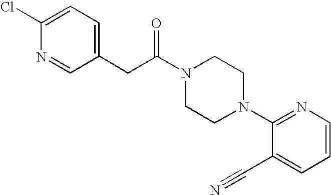 | 2-{4-[2-(6-Chloro-pyridin- 3-yl)-acetyl]-piperazin-1- yl}-nicotinonitrile | 800 mg, 80% |
| 16.11 | 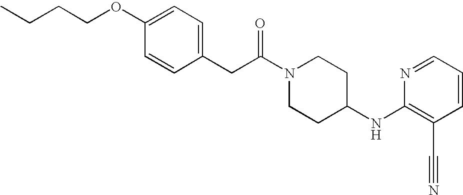 | 2-{1-[2-(4-Butoxy- phenyl)-acetyl]-piperidin- 4-ylamino}-nicotinonitrile | 300 mg, 39% |
| NMR | ¹H NMR (300 MHz, CDCl₃): δ(ppm) 8.05 (dd, 1H), 7.45 (dd, 1H), 6.97 | | |
| | (d, 2H), 6.66 (d, 2H), 6.42 (m, 1H), 4.76 (d, 1H), 4.43 (d, 1H), 3.98 (m, | | |
| | 1H), 3.74 (m, 3H), 3.50 (s, 2H), 2.94 (m, 1H), 2.61 (m, 1H), 1.82 (m, | | |
| | 2H), 1.56 (m, 2H), 1.22 (m, 3H), 0.95 (m, 1H), 0.77 (t, 3H). | | |
| 16.12 | 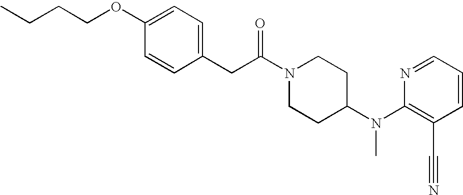 | 2-({1-[2-(4-Butoxy- phenyl)-acetyl]-piperidin- 4-yl}-methyl-amino)- nicotinonitrile | 420 mg, 44% |
| 16.13 | 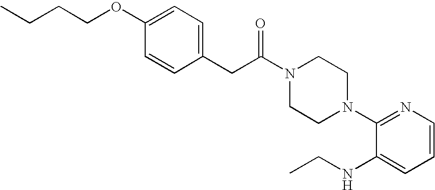 | 2-(4-Butoxy-phenyl)-1-[4- (3-ethylamino-pyridin-2- yl)-piperazin-1-yl]- ethanone | 380 mg, 40% |
| NMR | ¹H NMR (300MHz, CDCl₃): δ(ppm) 7.48 (dd, 1H), 6.97 (d, 2H), 6.66 | | |
| | (m, 4H), 3.98 (bs, 1H), 3.74 (t, 2H), 3.57 (m, 2H), 3.51 (s, 2H), 3.39 (m, | | |
| | 2H), 2.91 (q, 2H), 2.82 (m, 2H), 2.75 (m, 2H), 1.56 (m, 2H), 1.27 (m, | | |
| | 2H), 1.08 (t, 3H), 0.77 (t, 3H). | | |
| 16.14 | 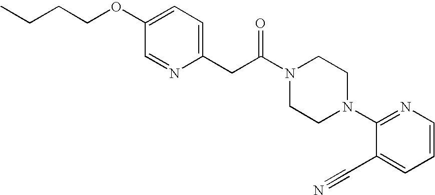 | 2-{4-[2-(5-Butoxy-pyridin- 2-yl)-acetyl]-piperazin-1- yl}-nicotinonitrile | 120 mg, 22% |

| | |
|---|---|
| NMR | ¹H NMR (300 MHz, CDCl₃): δ(ppm) 8.12 (dd, 1H), 7.98 (d, 1H), 7.57 |
| | (dd, 1H), 7.06 (m, 1H), 6.94 (m, 1H), 6.59 (m, 1H), 3.76 (t, 2H), 3.69 (s, |
| | 2H), 3.56 (m, 4H), 3.39 (m, 4H), 1.55 (m, 2H), 1.27 (m, 2H), 0.75 (t, |
| | 3H). |

### Example 17.1

### 1-(3-Nitro-pyridin-2-yl)-piperazine

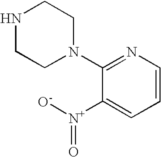

A mixture of 2-chloro-3-nitro-pyridine (2.0 g, 0.013 mol), piperazine (5.43 g, 0.063 mol) and acetonitrile (50 mL) was heated at reflux overnight. The reaction mixture was cooled to room temperature, diluted with ethyl acetate, washed with water and washed with brine. The organic layer was separated, dried over sodium sulfate, filtered and concentrated to give 1-(3-nitro-pyridin-2-yl)-piperazine (2.5 g, 95%). The product was used without further purification.

### Example 18.1

### 4-(3-Amino-pyridin-2-yl)-piperazine-1-carboxylic acid tert-butyl ester

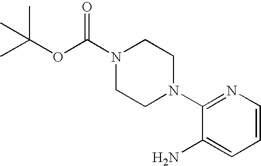

A mixture of 4-(3-nitro-pyridin-2-yl)-piperazine-1-carboxylic acid tert-butyl ester (4.0 g, 12.97 mmol), palladium (on activated carbon, 500 mg) and ethanol (150 mL) was shaken under 20 psi of hydrogen for 2 hours. The reaction mixture was filtered through Diatomaceous earth and concentrated to give 4-(3-amino-pyridin-2-yl)-piperazine-1-carboxylic acid tert-butyl ester (2.5 g, 69%). GC/MS m/z 278, 205, 134, 148, 109, 93, 57.

### Example 19.1

### 2-{4-[2-(4-Butylamino-phenyl)-acetyl]-piperazin-1-yl}-nicotinonitrile

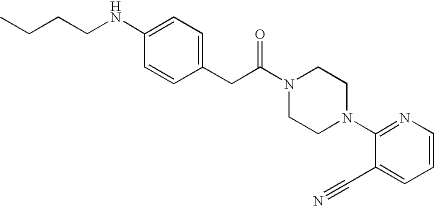

A mixture of 2-{4-[2-(4-amino-phenyl)-acetyl]-piperazin-1-yl}-nicotinonitrile (1.02 g, 3.17 mmol), 1-bromo-butane (0.34 mL, 3.16 mmol), cesium carbonate (1.04 g, 3.19 mmol) and N,N-dimethylformamide (30 mL) was stirred at 90° C. overnight. The reaction mixture was cooled to room temperature, water was added and the mixture was extracted with ethyl acetate. The combined organic layer was washed with water, washed with brine, dried over sodium sulfate, filtered and concentrated. The residue was purified on silica gel to give 2-{4-[2-(4-butylamino-phenyl)-acetyl]-piperazin-1-yl}-nicotinonitrile (850 mg, 71%). ¹H NMR (300 MHz, CDCl₃): δ(ppm) 8.14 (dd, 1H), 7.58 (dd, 1H), 6.88 (d, 2H), 6.60 (m, 1H), 6.37 (d, 2H), 3.59 (m, 2H), 3.45 (m, 7H), 3.32 (m, 2H), 2.90 (t, 2H), 1.40 (m, 2H), 1.22 (m, 2H), 0.75 (t, 3H).

In a similar manner the following compounds were synthesized:

| Example | Structure | Name | Yield |
|---|---|---|---|
| 19.2 | 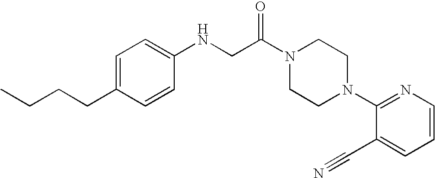 | 2-{4-[2-(4-Butyl- phenylamino)-acetyl- piperazin-1-yl}- nicotinonitrile | |
| NMR | ¹H NMR (300 MHz, CDCl₃): δ(ppm) 8.19 (dd, 1H), 7.63 (dd, 1H), 6.84 | | |
| | (d, 2H), 6.66 (m, 1H), 6.40 (d, 2H), 4.58 (bs, 1H), 3.73 (m, 2H), 3.65 (m, | | |
| | 2H), 3.54 (m, 4H), 3.44 (m, 2H), 2.32 (t, 2H), 1.35 (m, 2H), 1.15 (m, | | |
| | 2H), 0.73 (t, 3H). | | |
| 19.3 | 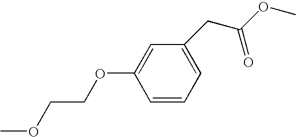 | [3-(2-Methoxy-ethoxy)- phenyl]-acetic acid methyl ester | 5.50 g, 81% |
| GC/MS | GC/MS m/z 224, 165, 107, 89, 59. | | |
| 19.4 | 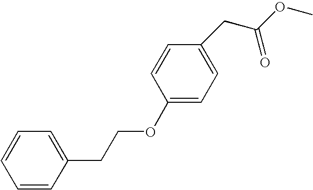 | (4-Phenethyloxy-phenyl)- acetic acid methyl ester | 750 mg, 23% |

| | |
|---|---|
| GC/MS | GC/MS m/z 270, 211, 105, 91, 77, 59. |

### Example 20.1

### 2-(4-{2-[4-(Butyl-methyl-amino)-phenyl]-acetyl}-piperazin-1-yl)-nicotinonitrile

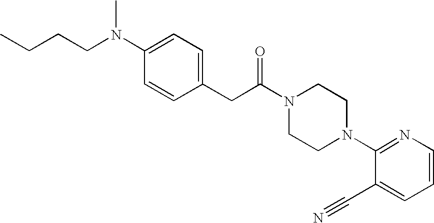

A mixture of 2-{4-[2-(4-butylamino-phenyl)-acetyl]-piperazin-1-yl}-nicotinonitrile (800 mg, 2.12 mmol), sodium hydride (60% dispersion in oil, 85 mg, 2.12 mmol), iodomethane (0.20 mL, 3.20 mmol) and N,N-dimethylformamide was stirred at 0° C. for 10 minutes and then at room temperature overnight. The reaction mixture was quenched with water and diluted with ethyl acetate. The organic layer was separated and sequentially washed with aqueous hydrochloric acid (0.1N), saturated aqueous sodium bicarbonate and brine. The organic layer was dried over sodium sulfate, filtered and concentrated. The residue was purified on silica gel to give 2-(4-{2-[4-(butyl-methyl-amino)-phenyl]-acetyl}-piperazin-1-yl)-nicotinonitrile. ¹H NMR (300 MHz, CDCl₃): δ(ppm) 8.14 (dd, 1H), 7.59 (dd, 1H), 6.90 (d, 2H), 6.60 (m, 1H), 6.44 (dd, 2H), 3.60 (m, 2H), 3.44 (m, 2H), 3.31 (m, 6H), 3.09 (t, 2H), 2.71 (s, 3H), 1.34 (m, 2H), 1.10 (m, 2H), 0.74 (t, 3H).

In a similar manner the following compounds were synthesized:

| Example | Structure | Name | Yield |
|---|---|---|---|
| 20.2 | 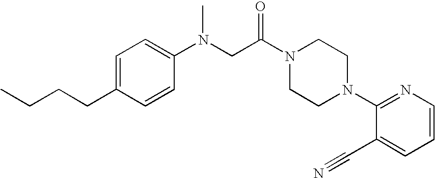 | 2-(4-{2-[(4-Butyl-phenyl)- methyl-amino}-acetyl}- piperazin-1-yl)- nicotinonitrile | |
| NMR | ¹H NMR (300 MHz, CDCl₃): δ(ppm) 8.18 (dd, 1H), 7.62 (dd, 1H), 6.87 | | |
| | (d, 2H), 6.65 (m, 1H), 6.50 (dd, 2H), 3.93 (s, 2H), 3.59 (m, 2H), 3.50 (m, | | |
| | 6H), 2.83 (s, 3H), 2.32 (t, 2H), 1.35 (m, 2H), 1.14 (m, 2H), 0.72 (t, 3H). | | |
| 20.3 | 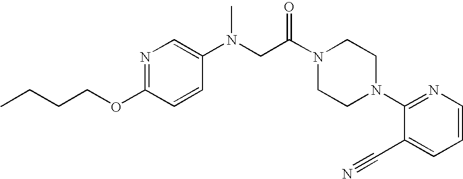 | 2-(4-{2-[(6-Butoxy- pyrdin-3-yl)-methyl- amino]-acetyl}-piperazin- 1-yl)-nicotinonitrile | 85 mg, 16% |

| | |
|---|---|
| NMR | ¹H NMR (300 MHz, CDCl₃): δ(ppm) 8.17 (dd, 1H), 7.61 (dd, 1H), 7.44 |
| | (d, 1H), 6.93 (dd, 1H), 6.64 (m, 1H), 6.45 (d, 1H), 3.98 (t, 2H), 3.87 (s, |
| | 2H), 3.53 (m, 8H), 2.80 (s, 3H), 1.52 (m, 2H), 1.25 (m, 2H), 0.75 (t, 3H). |

### Example 21.1

### 2-(4-{2-[4-(Pyridin-4-ylmethoxy)-phenyl]-acetyl}-piperazin-1-yl)-nicotinonitrile

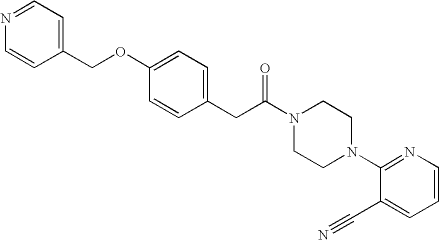

A mixture of 2-{4-[2-(4-hydroxy-phenyl)-acetyl]-piperazin-1-yl}-nicotinonitrile (Example 1.4) (500 mg, 1.55 mmol), sodium hydride (75 mg, 3.12 mmol), 4-bromomethyl-pyridine hydrobromide (393 mg, 1.55 mmol) and N,N-dimethylformamide (20 mL) was stirred at 90° C. The reaction mixture was cooled to room temperature, quenched with water and diluted with ethyl acetate. The organic layer was separated, washed with water, washed with brine, dried over sodium sulfate, filtered and concentrated. The residue was purified on silica gel to give 2-(4-{2-[4-(pyridin-4-ylmethoxy)-phenyl]-acetyl}-piperazin-1-yl)-nicotinonitrile (115 mg, 18%). ¹H NMR (300 MHz, CDCl₃): δ(ppm) 8.35 (bs, 2H), 8.08 (dd, 1H), 7.52 (dd, 1H), 7.10 (m, 2H), 6.95 (d, 2H), 6.67 (d, 2H), 6.55 (m, 1H), 4.82 (s, 2H), 3.53 (m, 2H), 3.48 (s, 2H), 3.38 (m, 4H), 3.27 (m, 2H),

### Example 22.1

### 2-Butoxy-5-nitro-pyridine

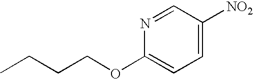

To a solution of butan-1-ol (6.52 mL, 71.34 mmol) in N,N-dimethylformamide (100 mL) at 0° C., was slowly added sodium hydride (60% dispersion in oil, 2.85 g, 71.25 mmol). The reaction mixture was stirred at 0° C. for 5 minutes and then 2-chloro-5-nitro-pyridine (5.66 g, 35.69 mmol) was added. The reaction mixture was stirred at room temperature until complete. The reaction mixture was quenched with water and diluted with ethyl acetate. The organic layer was separated, washed with water, washed with brine, dried over sodium sulfate, filtered and concentrated to give 2-butoxy-5-nitro-pyridine (4.52 g, 65%). GC/MS m/z 196, 167, 140, 124, 95, 77, 56.

### Example 23.1

### 6-Butoxy-pyridin-3-ylamine

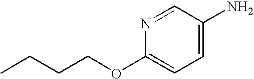

A mixture of 2-butoxy-5-nitro-pyridine (4.52 g, 23.04 mmol), palladium (on activated carbon, 150 mg) and ethanol was shaken under 20 atm of hydrogen for 2 hours. The reaction mixture was filtered through Diatomaceous earth and concentrated to give 6-butoxy-pyridin-3-ylamine (3.56 g, 93%). GC/MS m/z 166, 123, 110, 93, 82, 54.

In a similar manner the following compounds were synthesized

| Example | Structure | Name | Yield |
|---|---|---|---|
| 23.2 | 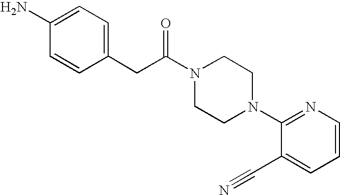 | 2-{4-[2-(4-Amino- phenyl)-acetyl]-piperazin- 1-yl}-nicotinonitrile | 1.02 g, 87 % |

| | |
|---|---|
| GC/MS | GC/MS m/z 321, 207, 133, 105, 77, 56. |

### Example 24.1

### 2-{4-[2-(6-Butoxy-pyridin-3-ylamino)-acetyl]piperazin-1-yl}-nicotinonitrile

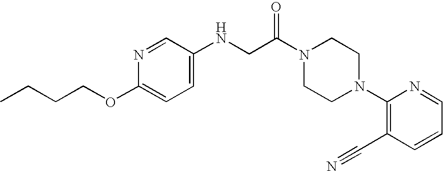

A mixture of 6-butoxy-pyridin-3-ylamine (3.54 g, 21.29 mmol), 2-[4-(2-bromo-acetyl)-piperazin-1-yl]-nicotinonitrile (6.57 g, 21.27 mmol), cesium carbonate (6.93 g, 21.27 mmol) and N,N-dimethylformamide was stirred at 60° C. The reaction mixture was cooled to room temperature, water was added and the mixture was extracted with ethyl acetate. The combined organic layer was washed with water, washed with brine, dried over sodium sulfate, filtered and concentrated. The residue was purified on silica gel to give 2-{4-[2-(6-butoxy-pyridin-3-ylamino)-acetyl]-piperazin-1-yl}-nicotinonitrile (2.02 g, 24%). ¹H NMR (300 MHz, CDCl₃): δ(ppm) 8.20 (m, 1H), 7.65 (dd, 1H), 7.37 (s, 1H), 6.85 (m, 1H), 6.68 (m, 1H), 6.44 (dd, 1H), 4.37 (s, 1H), 4.01 (t, 2H), 3.69 (m, 4H), 3.56 (m, 4H), 3.46 (m, 2H), 1.54 (m, 2H), 1.28 (m, 2H), 0.78 (t, 3H).

### Example 25.1

### 3-Chloro-isonicotinonitrile

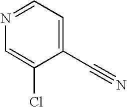

A mixture of 1-oxy-isonicotinonitrile (5.0 g, 0.042 mol), phosphorus pentachloride (12.0 g, 0.059 mol) and phosphorus oxychloride (20 mL) was heated at 120-130° C. for 2 hours. The reaction mixture was cooled, poured into ice and neutralized by the addition of solid sodium bicarbonate. The mixture was extracted with diethyl ether and the combined organic layer was dried over sodium sulfate, filtered and concentrated. The residue was purified on silica gel to give 3-chloro-isonicotinonitrile (1.2 g, 21%). ¹H NMR (300 MHz, CDCl₃): δ(ppm) 8.65 (s, 1H), 8.52 (d, 1H), 7.40 (d, 1H).

### Example 26.1

### 3-piperazin-1-yl-isonicotinonitrile

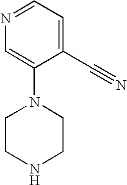

A mixture of 3-chloro-isonicotinonitrile (12 g, 8.66 mmol), piperazine (7.49 g, 8.69 mmol) and acetonitrile (50 mL) was heated at reflux overnight. The reaction mixture was cooled to room temperature, diluted with ethyl acetate, washed with water and washed with brine. The organic layer was dried over sodium sulfate, filtered and concentrated to give 3-piperazin-1-yl-isonicotinonitrile (1.5 g, 92%). The product was used without further purification.

### Example 27.1

### 2-{4-[2-(6-Butoxy-pyridin-3-yl)-acetyl]-piperazin-1-yl}-nicotinonitrile

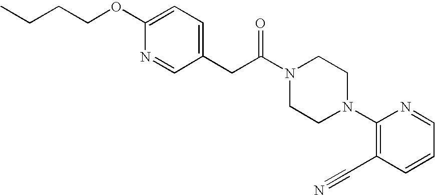

A mixture of 2-{4-[2-(6-chloro-pyridin-3-yl)-acetyl]-piperazin-1-yl}-nicotinonitrile (200 mg, 0.58 mmol), n-butanol (0.21 mL, 2.30 mmol), bisnaphthyl-di-tert-butyl palladium (46 mg, 0.11 mmol), cesium carbonate (190 mg, 0.58 mmol) and palladium acetate (26 mg, 0.11 mmol) was heated at 100° C. for 2 hours. The reaction mixture was concentrated and the residue was purified on silica gel using chloroform:methanol (95:5) to give 2-{4-[2-(6-butoxy-pyridin-3-yl)-acetyl]-piperazin-1-yl}-nicotinonitrile (150 mg, 67%).

### Example 28.1

### (4-Butoxy-phenyl)-carbamic acid tert-butyl ester

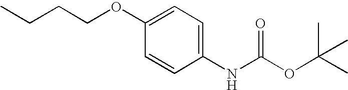

A mixture of (4-hydroxy-phenyl)-carbamic acid tert-butyl ester (2.0 g, 9.56 mmol), 1-bromo-butane (1.54 mL, 14.31 mmol), cesium carbonate (4.67 g, 14.33 mmol) and N,N-dimethylformamide (50 mL) was stirred at 60° C. for 3 hours. The reaction mixture was cooled to room temperature and water was added. The mixture was extracted with ethyl acetate and the combined organic layer was washed with brine, dried over sodium sulfate, filtered and concentrated to give (4-butoxy-phenyl)-carbamic acid tert-butyl ester (2.7 g). The product was used without further purification.

### Example 29.1

### 2-{4-[2-(4-Butoxy-phenylamino)-acetyl]-piperazin-1-yl}-nicotinonitrile

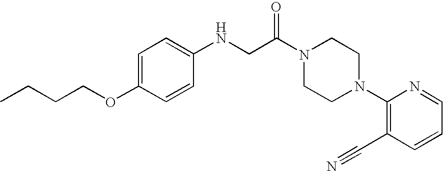

(4-Butoxy-phenyl)-carbamic acid tert-butyl ester (1.19 g, 4.48 mmol) was stirred in a trifluoroacetic acid:dichloromethane solution (50:50, 30 mL) for 1 hour. The reaction mixture was concentrated and the residue was dissolved in N,N-dimethylformamide (30 mL). To the solution was then added 2-[4-(2-chloro-acetyl)-piperazin-1-yl]-nicotinonitrile (1.2 g, 4.52 mmol) and cesium carbonate (1.47 g, 4.51 mmol). The reaction mixture was heated at 70° C. for 1 hour and then cooled to room temperature. The reaction mixture was diluted with ethyl acetate, washed with water and washed with brine. The organic layer was separated, dried over sodium sulfate, filtered and concentrated. The residue was purified on silica gel using hexanes:ethyl acetate (70:30) to give 2-{4-[2-(4-butoxy-phenylamino)-acetyl]-piperazin-1-yl}-nicotinonitrile (500 mg, 28%). ¹H NMR (300 MHz, CDCl₃): δ(ppm) 8.17 (dd, 1H), 7.63 (dd, 1H), 6.62 (m, 3H), 6.41 (d, 2H), 3.68 (m, 6H), 3.52 (m, 4H), 3.44 (m, 2H), 1.54 (m, 2H), 1.27 (m, 2H), 0.77 (t, 3H).

### Example 30.1

### 2-(Piperidin-4-ylamino)-nicotinonitrile

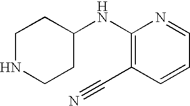

4-(3-Cyano-pyridin-2-ylamino)-piperidine-1-carboxylic acid tert-butyl ester (1.3 g, 4.30 mmol), was stirred in a trifluoroacetic acid:dichloromethane solution (50:50) for 1 hour. The reaction mixture was concentrated and the residue was dissolved in chloroform and basified with saturated aqueous potassium carbonate. The organic layer was separated, dried over sodium sulfate, filtered and concentrated to give 2-(piperidin-4-ylamino)-nicotinonitrile (400 mg, 46%).

### Example 31.1

### 4-(3-Cyano-pyridin-2-ylamino)-piperidine-1-carboxylic acid tert-butyl ester

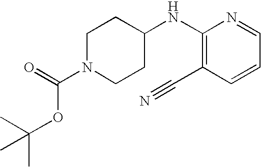

A mixture of 4-amino-piperidine-1-carboxylic acid tert-butyl ester (2.0 g, 9.99 mmol), 2-chloro-nicotinonitrile (1.38 g, 9.96 mmol), potassium carbonate (1.38 g, 10.00 mmol) and methyl sulfoxide (30 mL) was heated at 100° C. for 12 hours. The reaction mixture was cooled to room temperature and then diluted with ethyl acetate, washed with water and washed with brine. The organic layer was separated, dried over sodium sulfate, filtered and concentrated. The product was purified on silica gel using hexanes:ethyl acetate (80:20) to give 4-(3-cyano-pyridin-2-ylamino)-piperidine-1-carboxylic acid tert-butyl ester (1.3 g, 43%).

### Example 32.1

### 4-[(3-Cyano-pyridin-2-yl)-methyl-amino]-piperidine-1-carboxylic acid tert-butyl ester

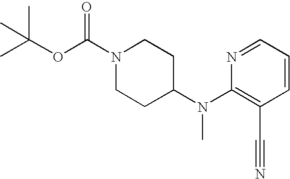

To a mixture of 4-(3-cyano-pyridin-2-ylamino)-piperidine-1-carboxylic acid tert-butyl ester (1.3 g, 4.3 mmol) and N,N-dimethylformamide (30 mL) at 0° C., was added sodium hydride (340 mg, 8.6 mmol). The reaction mixture was stirred at 0° C. for 10 minutes and then iodomethane (1.22 g, 8.6 mmol) was added. The reaction mixture was stirred at room temperature overnight and then diluted with ethyl acetate, washed with water and washed with brine. The organic layer was separated, dried over sodium sulfate, filtered and concentrated to give 4-[(3-cyano-pyridin-2-yl)-methyl-amino]-piperidine-1-carboxylic acid tert-butyl ester (1.3 g, 96%). The product was used without further purification.

### Example 33.1

### 4-(3-Nitro-pyridin-2-yl)-piperazine-1-carboxylic acid tort-butyl ester

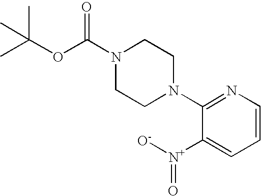

A mixture of 1-(3-nitro-pyridin-2-yl)-piperazine (2.5 g, 0.012 mol), di-tort-butyl dicarbonate (2.6 g, 0.012 mol) and tetrahydrofuran (50 mL) was stirred at room temperature for 2 hours. The reaction mixture was concentrated and the residue purified on silica gel using hexanes:ethyl acetate (90:10) to give 4-(3-nitro-pyridin-2-yl)-piperazine-1-carboxylic acid tert-butyl ester (3.0 g, 81%).

### Example 34.1

### 4-(3-Ethylamino-pyridin-2-yl)-piperazine-1-carboxylic acid tert-butyl ester

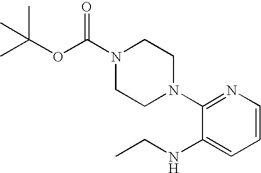

To a mixture of 4-(3-amino-pyridin-2-yl)-piperazine-1-carboxylic acid tert-butyl ester (2.0 g, 7.18 mmol), acetaldehyde (0.81 mL, 0.014 mol) and methanol (30 mL) was added sodium cyanoborohydride (1.36 g, 0.022 mol). The reaction mixture was stirred at room temperature for 2 days and then diluted with ethyl acetate, washed with water and washed with brine. The organic layer was separated, dried over sodium sulfate, filtered and concentrated. The residue was purified on silica gel using hexanes:ethyl acetate (80:20) to give 4-(3-ethylamino-pyridin-2-yl)-piperazine-1-carboxylic acid tert-butyl ester (2.0 g, 91%). GC/MS m/z 306, 223, 162, 150, 137, 120, 57.

### Example 35.1

### 5-Butoxy-2-methyl-pyridine

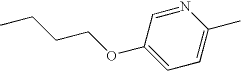

A mixture of 6-methyl-pyridin-3-ol (0.046 mol), 1-bromo-butane (4.93 mL, 0.046 mol), potassium hydroxide (5.13 g, 0.092 mol) and N,N-dimethylformamide was stirred at 70° C. for 1.5 hours. The reaction mixture was cooled to room temperature, diluted with ethyl acetate and washed with water and brine. The organic layer was dried over sodium sulfate, filtered and concentrated. The residue was purified on silica gel using hexanes:ethyl acetate (80:20) to give 5-butoxy-2-methyl-pyridine (5.0 g, 66%). GC/MS m/z 165, 109, 80, 53.

### Example 36.1

### 5-Butoxy-2-methyl-pyridine 1-oxide

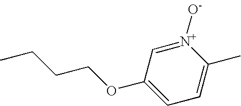

A solution of 5-butoxy-2-methyl-pyridine (5.0 g, 0.030 mol), 3-chloroperoxybenzoic acid (8.35 g, 0.048 mol) and dichloromethane (150 mL), was heated at 40° C. overnight. The reaction mixture was cooled to room temperature, diluted with dichloromethane and washed with aqueous potassium carbonate. The organic layer was separated, washed with brine, dried over sodium sulfate, filtered and concentrated to give 5-butoxy-2-methyl-pyridine 1-oxide (5.12 g, 91%). The product was used without further purification.

### Example 37.1

### Acetic acid 5-butoxy-1-oxy-pyridin-2-ylmethyl ester

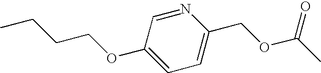

A mixture of 5-butoxy-2-methyl-pyridine 1-oxide (5.12 g, 0.028 mol) and acetic anhydride (50 mL) was heated at 120° C. for 3 hours. The reaction mixture was concentrated and the residue dissolved in methanol. Activated carbon was added and the suspension was stirred at room temperature. The mixture was filtered through Diatomaceous earth and the filtrate was concentrated to give acetic acid 5-butoxy-1-oxy-pyridin-2-ylmethyl ester (5.01 g, 74%). The product was used without further purification.

### Example 38.1

### 5-Butoxy-2-chloromethyl-pyridine

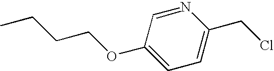

To a solution of acetic acid 5-butoxy-1-oxy-pyridin-2-ylmethyl ester (3.50 g, 0.019 mol) in dichloromethane (50 mL) was added thionyl chloride (14.1 mL, 0.194 mol). The reaction mixture was stirred at room temperature for 3 hours and then concentrated to give 5-butoxy-2-chloromethyl-pyridine (3.62 g, 94%). The product was used as is without further purification. GC/MS m/z 199, 143, 108, 78, 51.

### Example 39.1

### (5-Butoxy-pyridin-2-yl)-acetonitrile

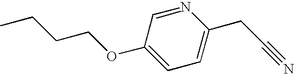

A mixture of 5-butoxy-2-chloromethyl-pyridine (3.0 g, 0.015 mol), sodium cyanide (2.21 g, 0.045 mol) and ethanol (80 mL) was heated at 80° C. for 4 hours. The reaction mixture was cooled to room temperature and diluted with water and ethyl acetate. The organic layer was separated, washed with brine, dried over sodium sulfate, filtered and concentrated to give (5-butoxy-pyridin-2-yl)-acetonitrile (450 mg, 16%). GC/MS m/z 190, 134, 106, 78, 57, 41.

### Example 40.1

### (5-Butoxy-pyridin-2-yl)-acetic acid ethyl ester

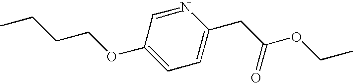

A mixture of (5-butoxy-pyridin-2-yl)-acetonitrile (450 mg, 2.36 mmol), concentrated hydrochloric acid (10 mL) and ethanol (50 mL) was heated at reflux overnight. The reaction mixture was cooled to room temperature, concentrated and the residue cooled to 0° C. Saturated aqueous sodium carbonate was added to pH 9. The resulting mixture was extracted with ethyl acetate and the combined organic layer was dried over sodium sulfate, filtered and concentrated to give (5-butoxy-pyridin-2-yl)-acetic acid ethyl ester (400 mg, 71%). GC/MS m/z 237, 165, 135, 108, 80, 52.

### Example 41.1

### (4-Butyl-phenyl)-methyl-amine

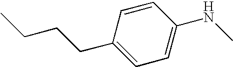

To a solution of 4-butyl-phenylamine (10.0 mL, 63.3 mmol) in tetrahydrofuran (150 mL) was added diisopropyl ethylamine (13.0 mL, 74.6 mmol). The reaction mixture was cooled to 0° C. and ethyl chloroformate (6.8 mL, 71.1 mmol) was added dropwise. The reaction mixture was stirred at 0° C. for 30 min and then at room temperature overnight. The reaction mixture was concentrated, diluted with ethyl acetate (350 mL) and washed with aqueous hydrochloric acid (1M, 100 mL). The aqueous layer was separated and extracted further with ethyl acetate (2×200 mL). The combined organic layer was washed with water (100 mL), washed with brine (100 mL), dried over sodium sulfate, filtered and concentrated. The residue was dissolved in tetrahydrofuran (110 mL) and cooled to 0° C. Lithium aluminum hydride (2M iii tetrahydrofuran, 63 mL, 126 mmol) was added over 5 minutes and the reaction mixture was then heated at reflux under nitrogen. After heating at reflux for 2 hours, the reaction mixture was cooled in an ice bath and quenched with the dropwise addition of water (5 mL), aqueous sodium hydroxide (15%, 5 mL) and water (15 mL) again. The resulting suspension was stirred, filtered and the solid rinsed with tetrahydrofuran (3×50 mL). The filtrate was concentrated and the residue purified on silica gel using hexanes:ethyl:acetate (95:5 to 90:10) to give (4-butyl-phenyl)-methyl-amine (8.92 g, 86%). ¹H NMR (300 MHz, CDCl₃): δ(ppm) 7.03 (d, 2H), 6.58 (d, 2H), 3.59 (bs, 1H), 2.83 (s, 3H), 2.52 (t, 2H), 1.57 (m, 2H), 1.35 (m, 2H), 0.93 (t, 3H).

### Example 42.1

### 4-{2-[(4-Butyl-phenyl)-methyl-amino]-acetyl}-3,4,5,6-tetrahydro-2H-[1,2′]bipyrazinyl-3′-carbonitrile

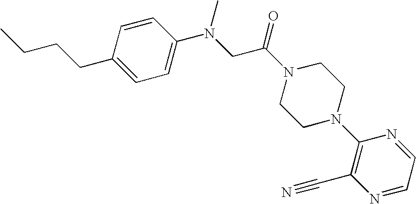

A mixture of (4-butyl-phenyl)-methyl-amine (68.5 mg, 0.42 mmol), potassium carbonate (105 mg, 0.76 mmol), acetonitrile (1.0 mL) and 4-(2-chloro-acetyl)-3,4,5,6-tetrahydro-2H-[1,2′]bipyrazinyl-3′-carbonitrile (100.5 mg, 0.38 mmol) was heated at 80° C. under nitrogen overnight. The reaction mixture was cooled to room temperature, diluted with water (5 mL) and extracted with ethyl acetate (3×10 mL). The combined organic layer was concentrated and the residue purified on silica gel using hexanes:ethyl acetate:dichloromethane (40:20:40) to give impure product. Purification on silica gel was repeated using hexanes:ethyl acetate:dichloromethane (45:10:45 to 40:20:40) to give impure product once again. The impure product was triturated with hexanes:diethyl ether (90:10, 3×3 mL) to give 4-{2-[(4-butyl-phenyl)-methyl-amino]-acetyl}-3,4,5,6-tetrahydro-2H-[1,2′]bipyrazinyl-3′-carbonitrile (44.1 mg, 30%). NMR (300 MHz, CDCl₃): δ(ppm) 8.30 (d, 1H), 8.10 (d, 1H), 7.07 (d, 2H), 6.69 (d, 2H), 4.11 (s, 2H), 3.76 (m, 8H), 3.01 (s, 3H), 2.52 (t, 2H), 1.55 (m, 2H), 1.33 (m, 2H), 0.92 (t, 3H).

### Example 43.1

### 2-((R)-3-Methyl-piperazin-1-yl)-nicotinonitrile

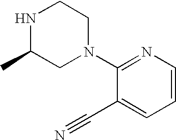

A mixture of (R)-2-methyl piperazine (507 mg, 5.06 mmol), 2-chloro-nicotinonitrile (1.05 g, 7.6 mmol), triethylamine (2 mL, 14.3 mmol) and tetrahydrofuran (8 mL) was heated at 80° C. overnight. The reaction mixture was cooled to room temperature and aqueous saturated sodium bicarbonate (75 mL) was added. The mixture was extracted with dichloromethane (3×200 mL) and the combined organic layer washed with water (75 mL), washed with brine (75 mL), dried over sodium sulfate, filtered and concentrated. The residue was purified on silica gel using dichloromethane: 2M ammonia in methanol (95:5) to give 2-((R)-3-methyl-piperazin-1-yl)-nicotinonitrile (964 mg, 94%). ¹H NMR (300 MHz, CDCl₃): δ(ppm) 8.34 (dd, 1H), 7.77 (dd, 1H), 6.74 (m, 1H), 4.27 (m, 2H), 3.08 (m, 4H), 2.70 (m, 1H), 1.15 (d, 3H).

In a similar manner the following compounds were synthesized:

| Example | Structure | Name | Yield |
|---|---|---|---|
| 43.2 | 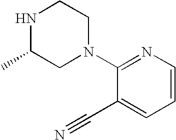 | 2-((S)-3-Methyl- piperazin- 1-yl)- nicotinonitrile | 910 mg, 89 % |

| | |
|---|---|
| NMR | ¹H NMR (300 MHz, CDCl₃): δ(ppm) 8.34 (dd, 1H), |
| | 7.77 (dd, 1H), 6.74 (m, 1H), 4.27 |
| | (m, 2H), 3.04 (m, 4H), |
| | 2.70 (m, 1H), 1.15 (d, 3H). |

### Example 44.1

### [(4-Butyl-phenyl)-methyl-amino]-acetic acid tert-butyl ester

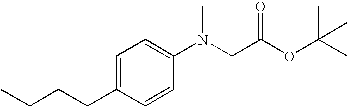

A mixture of (4-butyl-phenyl)-methyl-amine (1.15 g, 7.06 mmol), bromo-acetic acid tert-butyl ester (1.33 mL, 9.0 mmol), tetrabutylammonium hydrogen sulfate (143 mg, 0.42 mmol), aqueous sodium hydroxide (50%, 1.4 mL) and toluene (7 mL) was heated at 85° C. overnight. The reaction mixture was cooled to room temperature and acidified with aqueous hydrochloric acid (1M, 30 mL). The mixture was extracted with dichloromethane (3×) and the combined organic layer was washed with water (50 mL), washed with brine (50 mL), dried over sodium sulfate, filtered and concentrated. The residue was purified on silica gel using hexanes:ethyl acetate (97.5:2.5 to 96.5:3.5) to give [(4-butyl-phenyl)-methyl-amino]-acetic acid tert-butyl ester (857.6 mg, 44%). NMR (300 MHz, CDCl₃): δ(ppm) 7.04 (d, 2H), 6.62 (d, 2H), 3.93 (s, 2H), 3.04 (s, 3H), 2.51 (t, 2H), 1.55 (m, 2H), 1.42 (s, 9H), 1.35 (m, 2H), 0.92 (t, 3H).

### Example 45.1

### 2-[(4-Butyl-phenyl)-methyl-amino]-propionic acid tert-butyl ester

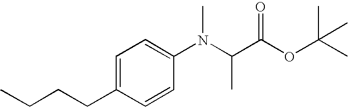

To a solution of (4-butyl-phenyl)-methyl-amine (500.2 mg, 3.06 mmol) in N,N-dimethylformamide at 0° C., was added sodium hydride (60% dispersion in oil, 155 mg, 3.87 mmol). The reaction mixture was stirred at 0° C. for 5 minutes and then 2-bromo-propionic acid tert-butyl ester (0.76 mL, 4.58 mmol) was added. The reaction mixture was stirred at 110° C. for 50 minutes. The reaction was not complete by TLC therefore additional 2-bromo-propionic acid tert-butyl ester (0.40 mL) was added. The reaction mixture was stirred at 110° C. for an additional 15 minutes and then cooled to room temperature. The reaction mixture was quenched by the addition of water (5 mL) and the resulting mixture was extracted with diethyl ether (350 mL). The organic layer was washed sequentially with aqueous hydrochloric acid (0.5N, 2×50 mL), water (5×50 mL) and brine (50 mL). The organic layer was dried over sodium sulfate, filtered and concentrated. The residue was purified on silica gel using hexanes:ethyl acetate (98:2 to 90:10) to give 2-[(4-butyl-phenyl)-methyl-amino]-propionic acid tert-butyl ester (380 mg, 42%). NMR (300 MHz, CDCl₃): δ(ppm) 7.05 (d, 2H), 6.73 (d, 2H), 4.35 (q, 1H), 2.88 (s, 3H), 2.52 (t, 2H), 1.55 (m, 2H), 1.43 (d, 3H), 1.41 (s, 3H), 1.34 (m, 2H), 0.92 (t, 3H).

### Example 46.1

### [(4-Butyl-phenyl)-methyl-amino]-acetic acid hydrochloride

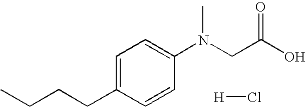

To a solution of [(4-butyl-phenyl)-methyl-amino]-acetic acid tert-butyl ester (853.9 mg, 3.08 mmol) in dichloromethane (8.5 mL) at 0° C., was added trifluoroacetic acid (8.5 mL). The reaction mixture was stirred at room temperature for 2.5 hours. The reaction mixture was concentrated and the residue was dissolved in 1,2-dichloroethane (5 mL). The mixture was concentrated and hydrochloric acid (2M in diethyl ether, 15 mL) was added. The mixture was concentrated and dichloromethane (10 mL) was added. The mixture was concentrated once again to give [(4-butyl-phenyl)-methyl-amino]-acetic acid hydrochloride (376 mg, 47%). NMR (300 MHz, CD₃OD): δ(ppm) 7.51 (bs, 2H), 7.36 (bs, 2H), 4.55 (bs, 2H), 3.30 (bs, 3H), 2.65 (t, 2H), 1.59 (m, 2H), 1.34 (m, 2H), 0.92 (t, 3H).

In a similar manner the following compounds were synthesized:

| Example | Structure | Name | Yield |
|---|---|---|---|
| 46.2 | 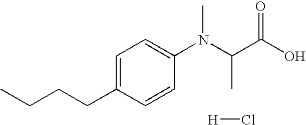 | 2-[(4-Butyl-phenyl)- methyl-amino]-propionic acid hydrochloride | Yellow foam solid, 273 mg, 78% |

| | |
|---|---|
| NMR | ¹H NMR (300 MHz, CD₃OD): δ(ppm) 7.52 (d, 2H), 7.4 (d, 2H), 4.67 (m, |
| | 1H), 3.31 (s, 3H), 2.68 (t, 2H), 1.61 (5, 2H), 1.30 (m, 2H), 0.94 (t, 3H). |

### Example 47.1

### 2-((R)-4-{2-[(4-Butyl-phenyl)-methyl-amino]-acetyl}-3-methyl-piperazin-1-yl)-nicotinonitrile

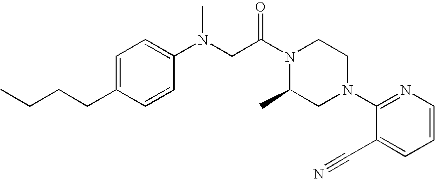

To a mixture of [(4-butyl-phenyl)-methyl-amino]-acetic acid hydrochloride (75.9 mg, 0.29 mmol), 2((R)-3-methyl-piperazin-1-yl)-nicotinonitrile (66.5 mg, 0.33 mmol), 1-ethyl-3-(3-dimethylaminopropyl)carbodiimide hydrochloride (56.9 mg, 0.30 mmol) and 1-hydroxybenzotriazole hydrate (41.9 mg, 0.31 mmol) was added N,N-dimethylformamide (0.5 mL) and triethylamine (0.06 mL, 0.43 mmol). The reaction mixture was stirred at room temperature overnight. The reaction mixture was diluted with ethyl acetate and sequentially washed with water, saturated aqueous sodium bicarbonate, water and brine. The organic layer was dried over sodium sulfate, filtered and concentrated. The residue was purified on silica gel using hexanes:ethyl acetate (65:35) to give 2-((R)-4-{2-[(4-butyl-phenyl)-methyl-amino]-acetyl}-3-methyl-piperazin-1-yl)-nicotinonitrile (27.0 mg, 23%) as a pale orange oil. ¹H NMR (300 MHz, CDCl₃): δ(ppm) 8.37 (dd, 1H), 7.81 (dd, 1H), 7.06 (d, 2H), 6.83 (m, 1H), 6.68 (d, 2H), 4.87 (bs, 0.5H), 4.49 (bd, 0.5H), 4.18 (m, 4.5H), 3.79 (bd, 0.5H), 3.62 (bt, 0.5H), 3.31 (dd, 1H), 3.10 (m, 1.5H), 3.01 (s, 3H), 2.52 (t, 2H), 1.55 (m, 2H), 1.33 (m, 5H), 0.92 (t, 3H).

In a similar manner the following compounds were synthesized:

| Example | Structure | Name | Yield |
|---|---|---|---|
| 47.2 | 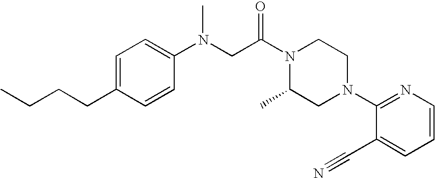 | 2-((S)-4-{2-[(4-Butyl- phenyl)-methyl-amino]- acetyl}-3-methyl- piperazin-1-yl)- nicotinonitrile | Orange- red oil, 19.0 mg, 16% |
| NMR | ¹H NMR (300 MHz, CDCl₃): δ(ppm) 8.37 (dd, 1H), 7.81 (dd, 1H), 7.06 | | |
| | (d, 2H), 6.83 (m, 1H), 6.68 (d, 2H), 4.87 (bs, 0.5H), 4.49 (bd, 0.5H), 4.18 | | |
| | (m, 4.5H), 3.79 (bd, 0.5H), 3.62 (bt, 0.5H), 3.31 (dd, 1H), 3.10 (m, 1.5H), | | |
| | 3.01 (s, 3H), 2.52 (t, 2H), 1.55 (m, 2H), 1.33 (m, 5H), 0.92 (t, 3H). | | |
| 47.3 | 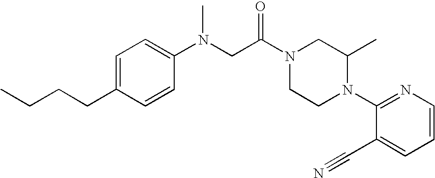 | 2-(4-{2-[(4-Butyl-phenyl)- methyl-amino]-acetyl}-2- methyl-piperazin-1-yl)- nicotinonitrile | 35.4 mg, 37% |
| NMR | ¹H NMR (300 MHz, CDCl₃): δ(ppm) 8.37 (d, 1H), 7.80 (dd, 1H), 7.07 (d, | | |
| | 2H), 6.80 (m, 1H), 6.68 (t, 2H), 4.66 (m, 1H), 4.52 (d, 0.5H), 4.28 (d, | | |
| | 0.5H), 4.10 (m, 3H), 3.94 (d, 0.5H), 3.72 (d, 0.5H), 3.55 (dd, 0.5H), 3.42 | | |
| | (m, 1.5H), 3.17 (dd, 0.5H), 3.02 (m, 3.5H), 2.52 (t, 2H), 1.54 (m, 2H), | | |
| | 1.29 (m, 5H), 0.92 (t, 3H). | | |
| 47.4 | 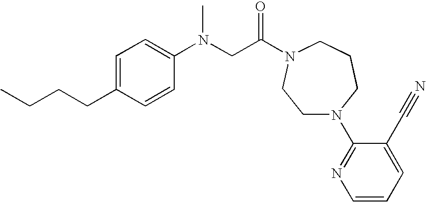 | 2-(4-{2-[(4-Butyl-phenyl)- methyl-amino]-acetyl}- [1,4]diazepan-1-yl)- nicotinonitrile | 23.5 mg, 20% |
| NMR | ¹H NMR (300 MHz, CDCl₃): δ(ppm) 8.31 (m, 1H), 7.75 (m, 1H), 6.99 | | |
| | (m, 2H), 6.69 (m, 1H), 6.57 (m, 2H), 3.98 (m, 6H), 3.82 (m, 2H), 3.55 | | |
| | (m, 2H), 2.95 (d, 3H), 2.48 (m, 2H), 2.10 (m, 1H), 2.00 (m, 1H), 1.53 (m, | | |
| | 2H), 1.33 (m, 2H), 0.92 (t, 3H). | | |
| 47.5 | 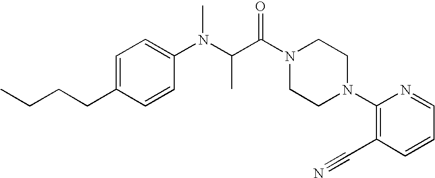 | 2-(4-{2-[(4-Butyl-phenyl)- methyl-amino]- propionyl}-piperazin-1- yl)-nicotinonitrile | 95.3 mg, 67% |

| | |
|---|---|
| NMR | ¹H NMR (300 MHz, CDCl₃): δ(ppm) 8.34 (dd, 1H), 7.79 (dd, 1H), 7.10 |
| | (d, 2H), 6.81 (m, 1H), 6.73 (d, 2H), 4.63 (q, 1H), 4.06 (m, 1H), 3.83 (m, |
| | 1H), 3.56 (m, 6H), 2.77 (s, 3H), 2.54 (t, 2H), 1.58 (m, 2H), 1.34 (m, 2H), |
| | 1.31 (d, 3H), 0.94 (t, 3H). |

### Example 48.1

### 2-(2-Methyl-piperazin-1-yl)-nicotinonitrile

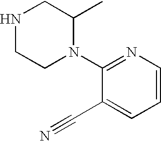

A mixture of 3-methyl-piperazine-1-carboxylic acid tert-butyl ester (1.00 g, 5.00 mmol), 2-chloro-nicotinonitrile (1.04 g, 7.48 mmol), triethylamine (2 mL) and tetrahydrofuran (8 mL) was heated at 80° C. overnight. The reaction mixture was cooled to room temperature and dichloromethane (300 mL) and aqueous saturated sodium bicarbonate (75 mL) were added. The aqueous mixture was separated and extracted further with dichloromethane (2×150 mL). The combined organic layer was washed with water (150 mL), washed with brine (100 mL), dried over sodium sulfate, filtered and concentrated. The residue was dissolved in dichloromethane (10 mL) and trifluoroacetic acid (10 mL) was added. The reaction mixture was stirred at room temperature for 2.5 hours and then concentrated. The residue was dissolved in 1,2-dichloroethane (5 mL) and the mixture was then concentrated. To the residue was added saturated aqueous sodium bicarbonate (150 mL). The mixture was extracted with dichloromethane (3×100 mL) and the combined organic layer was washed with water (50 mL), washed with brine (50 mL), dried over sodium sulfate, filtered and concentrated. The residue was purified on silica gel using dichloromethane:2M ammonia in methanol (95:5 to 92.5:7) to give 2-(2-methyl-piperazin-1-yl)-nicotinonitrile (48 mg, 5%). ¹H NMR (300 MHz, CDCl₃): δ(ppm) 8.33 (dd, 1H), 7.76 (dd, 1H), 6.71 (m, 1H), 4.62 (m, 1H), 4.03 (bd, 1H), 3.35 (m, 1H), 3.10 (m, 2H), 2.90 (m, 2H), 1.75 (bs, 1H), 1.34 (d, 3H).

### Example 49.1

### N-(4-Butyl-phenyl)-methanesulfonamide

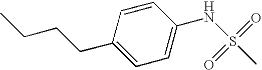

To a solution of 4-butyl-phenylamine (1.58 mL, 10.0 mmol) in dichloromethane (20 mL) at 0° C., was added pyridine (0.91 mL, 11.2 mmol) followed by methane sulfonyl chloride (0.87 mL, 11.2 mmol). The reaction mixture was stirred at 0° C. for 10 minutes and then at room temperature overnight. The reaction mixture was diluted with ethyl acetate (450 mL) and water (50 mL). The organic layer was separated, washed with water (50 mL), washed with brine (50 mL), dried over sodium sulfate, filtered and concentrated. The residue was purified on silica gel using dichloromethane to give N-(4-butyl-phenyl)-methanesulfonamide (2.21 g, 97%) as an off-white solid. ¹H NMR (300 MHz, CDCl₃): δ(ppm) 7.16 (s, 4H), 6.66 (s, 1H), 3.00 (s, 3H), 2.60 (t, 2H), 1.59 (m, 2H), 1.35 (m, 2H), 0.93 (t, 3H).

### Example 50.1

### N-(4-Butyl-phenyl)-N-{2-[4-(3-cyano-pyridin-2-yl)-piperazin-1-yl]-2-oxo-ethyl}-methanesulfonamide

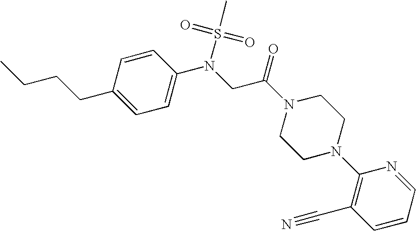

A mixture of 2-[4-(2-chloro-acetyl)-piperazin-1-yl]-nicotinonitrile (90.8 mg, 0.343 mmol), N-(4-butyl-phenyl)-methanesulfonamide (69.2 mg, 0.304 mmol), potassium carbonate (126 mg, 0.91 mmol), potassium iodide (101 mg, 0.61 mmol) and acetonitrile (2 mL) was heated at 80° C. overnight. The reaction mixture was cooled to room temperature, diluted with ethyl acetate (20 mL) and filtered. The filtrate was diluted with ethyl acetate (125 mL), washed with water (25 mL), washed with brine, dried over sodium sulfate, filtered and concentrated. The residue was purified on silica gel using hexanes:ethyl acetate (65:35 to 50:50) to give N-(4-butyl-phenyl)-N-{2-[4-(3-cyano-pyridin-2-yl)-piperazin-1-yl]-2-oxo-ethyl}-methanesulfonamide (84.7 mg, 61%). ¹H NMR (300 MHz, CDCl₃): δ(ppm) 8.37 (dd, 1H), 7.81 (dd, 1H), 7.47 (d, 2H), 7.19 (d, 2H), 6.84 (m, 1H), 4.58 (s, 2H), 3.78 (m, 2H), 3.70 (m, 4H), 3.58 (m, 2H), 3.18 (s, 3H), 2.61 (t, 2H), 1.58 (m, 2H), 1.34 (m, 2H), 0.93 (t, 3H).

In a similar manner the following compounds were synthesized:

| Example | Structure | Name | Yield |
|---|---|---|---|
| 50.2 | 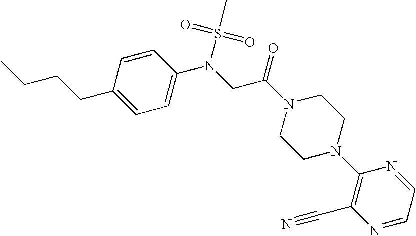 | N-(4-Butyl-phenyl)-N-[2- (3′-cyano-2,3,5,6- tetrahydro- [1,2′]bipyrazinyl-4-yl)-2- oxo-ethyl]- methanesulfonamide | 137 mg, 98% |

| | |
|---|---|
| NMR | ¹H NMR (300 MHz, CDCl₃): δ(ppm) 8.29 (d, 1H), 8.10 (d, 1H), 7.45 (d, |
| | 2H), 7.19 (d, 2H), 4.58 (s, 2H), 3.80 (m, 6H), 3.61 (m, 2H), 3.17 (s, 3H), |
| | 2.60 (t, 2H), 1.58 (m, 2H), 1.34 (m, 2H), 0.92 (t, 3H). |

### Example 51.1

### 2-(4-{2-[4-(Pyridin-2-ylmethoxy)-phenyl]-acetyl}-piperazin-1-yl)-nicotinonitrile

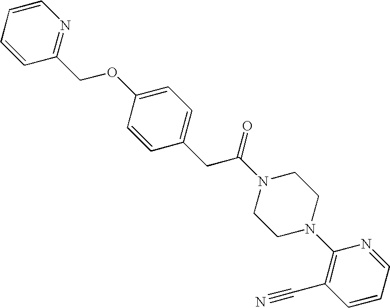

To a mixture of 2-{4-[2-(4-hydroxy-phenyl)-acetyl]-piperazin-1-yl}-nicotinonitrile (50.0 mg, 0.155 mmol) in N,N-dimethylformamide (5 mL) was added sodium hydride (60% dispersion in oil, 12.4 mg, 0.310 mmol) followed by 2-bromomethylpyridine (32.3 mg, 0.155 mmol). The reaction mixture was heated at 90° C. for 24 hours and then at room temperature overnight. The reaction mixture was quenched by the addition of water and the resulting mixture was extracted with ethyl acetate (3×). The combined organic layer was washed with water, washed with brine, dried over sodium sulfate, filtered and concentrated. The residue was purified on silica gel using dichloromethane:ethyl acetate (50:50 to 0:100) to give 2-(4-{2-[4-(pyridin-2-ylmethoxy)-phenyl]-acetyl}-piperazin-1-yl)-nicotinonitrile (40.7 mg, 63%) as a yellow oil. NMR (300 MHz, CDCl₃): δ(ppm) 8.59 (d, 1H), 3.34 (dd, 1H), 7.79 (dd, 1H), 7.71 (m, 1H), 7.51 (d, 1H), 7.20 (m, 3H), 6.95 (d, 2H), 6.81 (m, 1H), 5.19 (s, 2H), 3.79 (m, 2H), 3.72 (s, 2H), 3.64 (m, 4H), 3.51 (m, 2H).

In a similar manner the following compounds were synthesized:

| Example | Structure | Name | Yield |
|---|---|---|---|
| 51.2 | 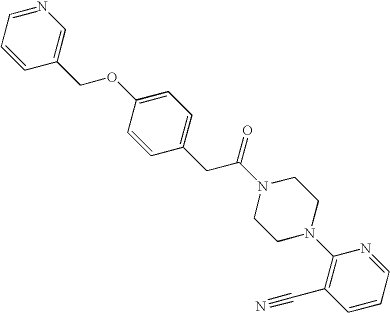 | 2-(4-{2-[4-(Pyridin-3- ylmethoxy)-phenyl]- acetyl}-piperazin-1-yl)- nicotinonitrile | White solid, 27.8 mg, 43% |

| | |
|---|---|
| NMR | ¹H NMR (300 MHz, CDCl₃): δ(ppm) 8.69 (s, 1H), 8.59 (d, 1H), 3.35 (dd, |
| | 1H), 7.79 (m, 2H), 7.34 (m, 1H), 7.20 (d, 2H), 6.95 (d, 2H), 6.82 (m, |
| | 1H), 5.07 (s, 2H), 3.81 (m, 2H), 3.74 (s, 2H), 3.64 (m, 4H), 3.53 (m, 2H). |

### Example 52.1

### 2-(4-{2-[4-(Thiazol-2-ylmethoxy)-phenyl]-acetyl}-piperazin-1-yl)-nicotinonitrile

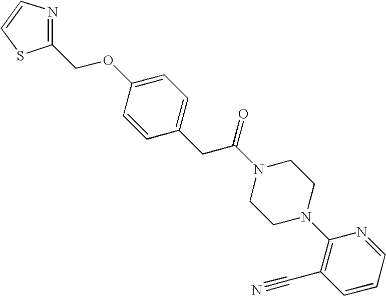

To a solution of 1,3-thiazole-2-ylmethanol (40.0 mg, 0.347 mmol) in dichloromethane (2 mL) was added triethylamine (72.6 uL, 0.521 mmol) followed by methanesulfonyl chloride (59.7 mg, 0.521 mmol). The reaction mixture was stirred at room temperature for 20 minutes. The reaction mixture was quenched by the addition of aqueous saturated sodium bicarbonate and then extracted with dichloromethane (3×). The combined organic layer was washed with water, washed with brine, dried over sodium sulfate, filtered and concentrated to give the mesylate intermediate. To a mixture of 2-{4-[2-(4-hydroxy-phenyl)-acetyl]-piperazin-1-yl}-nicotinonitrile (40.0 mg, 0.124 mmol) in N,N-dimethylformamide (2 mL) was added sodium hydride (60% dispersion in oil, 8.2 mg, 0.130 mmol). The reaction mixture was stirred for 20 minutes at room temperature under nitrogen. To the reaction mixture was then added a solution of the mesylate intermediate in N,N-dimethylformamide (2 mL). The reaction mixture was stirred at 70° C. for 3.5 hours, cooled to room temperature and then quenched by the addition of aqueous saturated sodium bicarbonate. The mixture was extracted with ethyl acetate (3×) and the combined organic layer was washed with water, washed with brine, dried over sodium sulfate, filtered and concentrated. The residue was purified on silica gel using hexanes:ethyl acetate (40:60 to 0:100) to give 2-(4-{2-[4-(thiazol-2-ylmethoxy)-phenyl]-acetyl}-piperazin-1-yl)-nicotinonitrile (41.3 mg, 79%) as an off-white solid. NMR (300 MHz, CDCl₃): δ(ppm) 8.69 (dd, 1H), 8.59 (m, 2H), 3.35 (d, 1H), 7.79 (d, 2H), 7.34 (d, 2H), 7.20 (m, 1H), 6.95 (s, 2H), 6.82 (m, 2H), 5.07 (s, 2H), 3.81 (m, 4H), 3.74 (m, 2H).

In a similar manner the following compounds were synthesized:

| Example | Structure | Name | Yield |
|---|---|---|---|
| 52.2 | 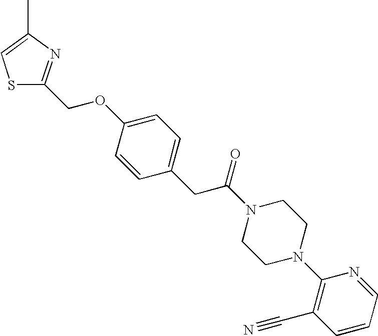 | 2-(4-{2-{4-(4-Methyl- thiazol-2-ylmethoxy)- phenyl]-acetyl}-piperazin- 1-yl)-nicotinonitrile | Clear oil, 28.3 mg, 54% |
| NMR | ¹H NMR (300 MHz, CDCl₃): δ(ppm) 8.34 (m, 1H), 7.78 (dd, 1H), 7.19 | | |
| | (d, 2H), 6.96 (d, 2H), 6.89 (s, 1H), 6.82 (m, 1H), 5.31 (s, 2H), 3.79 (m, | | |
| | 2H), 3.72 (s, 2H), 3.63 (m, 4H), 3.52 (m, 2H), 2.46 (s, 3H). | | |
| 52.3 | 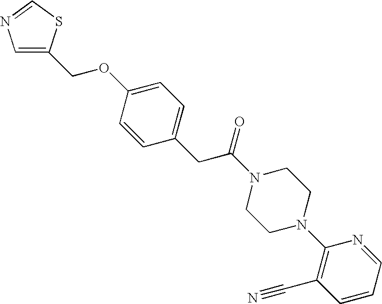 | 2-(4-{2-[4-(Thiazol-5- ylmethoxy)-phenyl]- aeetyl}-piperazin-1-yl)- nicotinonitrile | White solid, 25.5 mg |

| | |
|---|---|
| NMR | ¹H NMR (300 MHz, CDCl₃): δ(ppm) 8.81 (s, 1H), 8.34 (dd, 1H), 7.89 (s, |
| | 1H), 7.78 (dd, 1H), 7.19 (d, 2H), 6.93 (d, 2H), 6.81 (m, 1H), 5.26 (s, 2H), |
| | 3.79 (m, 2H), 3.72 (s, 2H), 3.62 (m, 4H), 3.52 (m, 2H). |

1. A compound of Formula: 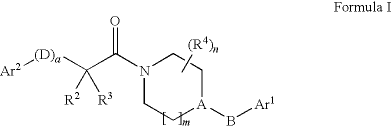
wherein:
Ar¹ is selected from the group consisting of aryl and heteroaryl, substituted with a CN group at the position alpha to the link with group B, further optionally-substituted with one or more substituents selected from the group consisting of alkyl, haloalkyl and halo;
Ar² is selected from the group consisting of aryl and heteroaryl, optionally substituted with one or more substituents selected from the group consisting of halo, alkyl, haloalkyl, alkoxy, haloalkoxy, NR⁵R⁶, O-alkylene-aryl, O-alkylene-heteroaryl, O-alkylene-O-alkyl, O-cycloalkyl, O-heterocycloalkyl, wherein any cyclic group may be further substituted with one or more substituents selected from the group consisting of alkyl and halo;
A is selected from the group consisting of C and N;
B is a bond when A is N and is group NR when A is C;
D is selected from the group consisting of NR¹ and O;
a is selected from the group consisting of 0 and 1;
m is selected from the group consisting of 1 and 2;
n is selected from the group consisting of 1, 2, 3 and 4;
R, R², R³, R⁵ and R⁶ are independently selected from the group consisting of H and alkyl;
R¹ is selected from the group consisting of H, alkyl, COR, CO₂R and SO₂R; and
R⁴ is selected from the group consisting of H, halo, CN, alkyl, haloalkyl, CH₂OR and CO₂R,
or a pharmaceutically acceptable salt, hydrate, solvate, optical isomer, or combination thereof.

2. A compound according to claim 1 wherein A is N and B is a bond, or a pharmaceutically acceptable salt thereof, or a hydrate thereof, or a solvate thereof, or an optical isomer thereof.

3. A compound according to claim 2 wherein a is 0, or a pharmaceutically acceptable salt thereof, or a hydrate thereof, or a solvate thereof, or an optical isomer thereof.

4. A compound according to claim 3 wherein m is 1, or a pharmaceutically acceptable salt thereof, or a hydrate thereof, or a solvate thereof, or an optical isomer thereof.

5. A compound according to claim 4 wherein Ar¹ is selected from the group consisting of phenyl, pyridyl and pyrazinyl, or a pharmaceutically acceptable salt thereof, or a hydrate thereof, or a solvate thereof, or an optical isomer thereof.

6. A compound according to claim 4 wherein Ar² is selected from the group consisting of phenyl and pyridyl, or a pharmaceutically acceptable salt thereof, or a hydrate thereof, or a solvate thereof, or an optical isomer thereof.

7. A compound selected from the group consisting of:
2-{4-[2-(4-Butoxy-phenyl)-acetyl]-piperazin-1-yl}-nicotinonitrile;
2-{4-[2-(4-Butoxy-phenyl)-acetyl]-piperazin-1-yl}-benzonitrile;
4-[2-(4-Butoxy-phenyl)-acetyl]-3,4,5,6-tetrahydro-2H-[1,2]bipyrazinyl-3′-carbonitrile;
2-{4-[2-(4-Hydroxy-phenyl)-acetyl]-piperazin-1-yl}-nicotinonitrile;
2-{4-[2-(4-Ethoxy-phenyl)-acetyl]-piperazin-1-yl}-nicotinonitrile;
2-{4-[2-(4-Butoxy-phenyl)-acetyl]-[1,4]diazepan-1-yl}-nicotinonitrile;
2-(4-Butoxy-phenyl)-1-[4-(3-chloro-pyridin-2-yl)-piperazin-1-yl]-ethanone;
2-{4-[2-(4-Isopropoxy-phenyl)-acetyl]-piperazin-1-yl}-nicotinonitrile;
2-(4-{2-[(4-Bromo-phenyl)-methyl-amino]-acetyl}-piperazin-1-yl)-nicotinonitrile;
2-{4-[2-(Methyl-p-tolyl-amino)-acetyl]-piperazin-1-yl}-nicotinonitrile;
2-{4-[2-(4-Phenoxy-phenyl)-acetyl]-piperazin-1-yl}-nicotinonitrile;
2-[4-(2-Phenoxy-acetyl)-piperazin-1-yl]-nicotinonitrile;
2-{4-[2-(4-Butyl-phenoxy)-acetyl]-piperazin-1-yl}-nicotinonitrile;
3-{4-[2-(4-Butoxy-phenyl)-acetyl]-piperazin-1-yl}-pyridine-2-carbonitrile;
2-{4-[2-(4-Benzyloxy-phenyl)-acetyl]-piperazin-1-yl}-nicotinonitrile;
2-{4-[2-(4-Propoxy-phenyl)-acetyl]-piperazin-1-yl}-nicotinonitrile;
2-(4-{2-[4-(Tetrahydro-furan-3-yloxy)-phenyl]-acetyl}-piperazin-1-yl)-nicotinonitrile;
2-{4-[2-(4-Nitro-phenyl)-acetyl]-piperazin-1-yl}-nicotinonitrile;
2-(4-{2-[4-(2-Methoxy-ethoxy)-phenyl]-acetyl}-piperazin-1-yl)-nicotinonitrile;
2-{4-[2-(4-Phenethyloxy-phenyl)-acetyl]-piperazin-1-yl}-nicotinonitrile;
3-{4-[2-(4-Butoxy-phenyl)-acetyl]-piperazin-1-yl}-isonicotinonitrile;
2-{4-[2-(4-Butoxy-phenyl)-acetyl]-piperazin-1-yl}-6-trifluoromethyl-nicotinonitrile;
2-{4-[2-(6-Chloro-pyridin-3-yl)-acetyl]-piperazin-1-yl}-nicotinonitrile;
2-{1-[2-(4-Butoxy-phenyl)-acetyl]-piperidin-4-ylamino}-nicotinonitrile;
2-({1-[2-(4-Butoxy-phenyl)-acetyl]-piperidin-4-yl}-methyl-amino)-nicotinonitrile;
2-(4-Butoxy-phenyl)-1-[4-(3-ethylamino-pyridin-2-yl)-piperazin-1-yl]-ethanone;
2-{4-[2-(5-Butoxy-pyridin-2-yl)-acetyl]-piperazin-1-yl}-nicotinonitrile;
2-{4-[2-(4-Butylamino-phenyl)-acetyl]-piperazin-1-yl}-nicotinonitrile;
2-{4-[2-(4-Butyl-phenylamino)-acetyl]-piperazin-1-yl}-nicotinonitrile;
2-(4-{2-[4-(Butyl-methyl-amino)-phenyl]-acetyl}-piperazin-1-yl)-nicotinonitrile;
2-(4-{2-[(4-Butyl-phenyl)-methyl-amino]-acetyl}-piperazin-1-yl)-nicotinonitrile;
2-(4-{2-[(6-Butoxy-pyridin-3-yl)-methyl-amino]-acetyl}-piperazin-1-yl)-nicotinonitrile;
2-(4-{2-[4-(Pyridin-4-ylmethoxy)-phenyl]-acetyl}-piperazin-1-yl)-nicotinonitrile;
2-{4-[2-(6-Butoxy-pyridin-3-ylamino)-acetyl]-piperazin-1-yl}-nicotinonitrile;
2-{4-[2-(6-Butoxy-pyridin-3-yl)-acetyl]-piperazin-1-yl}-nicotinonitrile;
2-{4-[2-(4-Butoxy-phenylamino)-acetyl]-piperazin-1-yl}-nicotinonitrile;
4-{2-[(4-Butyl-phenyl)-methyl-amino]-acetyl}-3,4,5,6-tetrahydro-2H-[1,2]bipyrazinyl-3′-carbonitrile;
2-((R)-4-{2-[(4-Butyl-phenyl)-methyl-amino]-acetyl}-3-methyl-piperazin-1-yl)-nicotinonitrile;
2-((S)-4-{2-[(4-Butyl-phenyl)-methyl-amino]-acetyl}-3-methyl-piperazin-1-yl)-nicotinonitrile;
2-(4-{2-[(4-Butyl-phenyl)-methyl-amino]-acetyl}-2-methyl-piperazin-1-yl)-nicotinonitrile;
2-(4-{2-[(4-Butyl-phenyl)-methyl-amino]-acetyl}-[1,4]diazepan-1-yl)-nicotinonitrile;
2-(4-{2-[(4-Butyl-phenyl)-methyl-amino]-propionyl}-piperazin-1-yl)-nicotinonitrile;
N-(4-Butyl-phenyl)-N-{2-[4-(3-cyano-pyridin-2-yl)-piperazin-1-yl]-2-oxo-ethyl}-methanesulfonamide;
N-(4-Butyl-phenyl)-N-[2-(3′-cyano-2,3,5,6-tetrahydro-[1,2]bipyrazinyl-4-yl)-2-oxo-ethyl]-methanesulfonamide;
2-(4-{2-[4-(Pyridin-2-ylmethoxy)-phenyl]-acetyl}-piperazin-1-yl)-nicotinonitrile;
2-(4-{2-[4-(Pyridin-3-ylmethoxy)-phenyl]-acetyl}-piperazin-1-yl)-nicotinonitrile;
2-(4-{2-[4-(Thiazol-2-ylmethoxy)-phenyl]-acetyl}-piperazin-1-yl)-nicotinonitrile;
2-(4-{2-[4-(4-Methyl-thiazol-2-ylmethoxy)-phenyl]-acetyl}-piperazin-1-yl)-nicotinonitrile, and
2-(4-{2-[4-(Thiazol-5-ylmethoxy)-phenyl]-acetyl}-piperazin-1-yl)-nicotinonitrile
or a pharmaceutically acceptable salt, hydrate, solvate or optical isomer, of any foregoing compound.

8. A pharmaceutical composition comprising a compound, or a pharmaceutically acceptable salt thereof, or a hydrate thereof, or a solvate thereof, or an optical isomer thereof, according to claim 1 and a pharmaceutically acceptable carrier or excipient.

9-11. (canceled)

12. A method for the treatment or prevention of neurological and psychiatric disorders associated with glutamate dysfunction in an animal in need of such treatment, comprising the step of administering to said animal a therapeutically effective amount of a compound, or a pharmaceutically acceptable salt thereof, or a hydrate thereof, or a solvate thereof, or an optical isomer thereof, according to claim 1.

13. A method for the treatment or prevention of neurological and psychiatric disorders associated with glutamate dysfunction in an animal in need of such treatment, comprising the step of administering to said animal a therapeutically effective amount of a pharmaceutical composition according to claim 8.

14. A method according to claim 12, wherein the disorder is schizophrenia.

15. A method according to claim 13, wherein the disorder is schizophrenia.
